(19) United States
(12) Patent Application Publication
(10) Pub. No.: US 2023/0263803 A1
(43) Pub. Date: Aug. 24, 2023

(54) METHODS OF PREVENTING OR TREATING COVID-19 AND RELATED VIRAL DISEASES OR DISORDERS

(71) Applicant: KINARUS AG, Basel (CH)

(72) Inventors: Alexander BAUSCH, Basel (CH); Matthew B. WRIGHT, Basel (CH)

(21) Appl. No.: 18/041,122

(22) PCT Filed: Aug. 9, 2021
(86) PCT No.: PCT/EP2021/072178

§ 371 Date: Feb. 9, 2023

(30) Foreign Application Priority Data
Aug. 11, 2020 (EP) 20190476.0
Feb. 11, 2021 (EP) 21156567.6
May 6, 2021 (EP) 21172474.5

(51) Int. Cl. A61K 31/519; A61K 31/4439; A61P 31/14; A61K 31/416; A61K 31/4418

(52) U.S. Cl. CPC A61K 31/519; A61K 31/4439; A61P 31/14; A61K 31/416; A61K 31/4418

(57) ABSTRACT

The present invention relates to a pharmaceutical combination comprising: (a) a PPAR agonist; (b) a p38 kinase inhibitor; and optionally (c) one or more pharmaceutically acceptable diluents, excipients or carriers for use in a method of preventing or treating viral diseases or disorders in a subject.

### THE FIELD OF THE INVENTION

The present disclosure provides combination therapy of a PPAR agonist and a P38 kinase inhibitor. The combination of PPAR agonists and a P38 kinase inhibitor may be useful in treating and/or preventing a viral disease or disorder in a subject. In certain embodiments, the viral infection is due to a cornonavirus, more the viral disease or disorder specifically is COVID-19.

### BACKGROUND OF THE INVENTION

In December 2019 a novel coronavirus was named as the severe acute respiratory syndrome coronavirus-2 (SARS-CoV-2, 2019-nCoV) due to its high homology (˜80%) to SARS-CoV, which caused acute respiratory distress syndrome (ARDS) and high mortality during 2002-2003. The outbreak of SARS-CoV-2 was considered to have originally started via a zoonotic transmission associated with the seafood market in Wuhan, China. Later it was recognized that human to human transmission played a major role in the subsequent outbreak. The disease caused by this virus was called Coronavirus disease 19 (COVID-19) and a pandemic was declared by the World Health Organization (WHO). COVID-19 has been impacting a large number of people worldwide, being reported in approximately 200 countries and territories. As of Apr. 7, 2020, around 1,400,000 cases worldwide have been reported according to the Center for Systems Science and Engineering (CSSE) at John Hopkins University.

SARS-CoV-2 virus primarily affects the respiratory system, although other organ systems are also involved. Lower respiratory tract infection related symptoms including fever, dry cough and dyspnea were reported in initial cases. It is now widely recognized that respiratory symptoms of COVID-19 are extremely heterogeneous, ranging from minimal symptoms to significant hypoxia leading to Acute Respiratory Distress Syndrome (ARDS). The time between the onset of symptoms and the development of ARDS is as short as 9 days, suggesting that the respiratory symptoms could progress rapidly. This disease, in addition to causing significant morbidity, has also proven to be fatal with approximately 140,000 deaths in the United States alone as of Jul. 7, 2020. Epidemiological studies have shown that mortalities are higher in the elder population and the incidence is much lower in children. Current medical management is largely supportive with no targeted therapy available. Several drugs including lopinavir-ritonavir, remdesivir, hydroxychloroquine, and azithromycin have been tested in clinical trials but none of them have yet been proven to be a useful therapy. A large number of countries have implemented social distancing and home isolation to mitigate further spread of the virus.

Coronaviruses are enveloped, positive-sense, single-stranded RNA viruses of ˜30 kb. They infect a wide variety of host species. Human coronaviruses such as 229E and NL63 are responsible for common cold. In contrast, SARS-CoV, Middle East respiratory syndrome coronavirus (MERS-CoV) and SARS-CoV-2 are classified as R coronaviruses.

The life cycle of the virus with the host consists of the following 5 steps: cell attachment, penetration, viral replication, biosynthesis, maturation and release or shedding. Once viruses bind to host receptors (attachment), they enter host cells through endocytosis or membrane fusion (penetration). Once viral contents are released inside the host cells, viral RNA enters the nucleus for replication. Viral mRNA is used to make viral proteins (biosynthesis). Then, new viral particles are made (maturation) and released (shed).

Shedding of SARS-CoV-2 from infected patients has led to rapid spread of the global pandemic. RNA has been identified by qRT-PCR in respiratory tract samples 1-2 days prior to symptom onset and can persist for 7-12 days in moderate cases and for up to 2 weeks in severe cases. In several cases with serial sampling, viral loads were shown to be highest soon after symptom onset. Patients with severe COVID-19 had a significantly higher viral load and a longer period of viral shedding than patients with mild cases. Prolonged viral RNA shedding has been reported from throat swabs for up to 37 days among adult patients, and in faeces for over 1 month after illness onset in children.

The symptom of patients infected with SARS-CoV-2 ranges from minimal symptoms to severe respiratory failure with multiple organ failure. Patients with severe disease were reported to have increased plasma concentrations of proinflammatory cytokines, including interleukin (IL)-6, IL-10, granulocyte-colony stimulating factor (G-CSF), monocyte chemoattractant protein 1 (MCP1), macrophage inflammatory protein (MIP)1α, and tumor necrosis factor (TNF)-α. More severe disease was associated with higher IL-6. The study of SARS-CoV showed that virus infected lung epithelial cells produced IL-8 in addition to IL-6. IL-8 is a well-known chemoattractant for neutrophils and T cells. Infiltration of a large number of inflammatory cells were observed in the lungs from severe COVID-19 patients.

There is a need to provide improved treatment methods to make available significant benefit for COVID-19 patients as well as patients infected with other types of viruses that primarily target the lungs.

### SUMMARY OF THE INVENTION

It has now unexpectedly been found that a pharmaceutical combination comprising a PPAR agonist, such as pioglitazone and a p38 inhibitor, e.g. a compound of formula I or II as defined herein below, such as pamapimod, is useful for preventing or treating viral infections such as COVID-19 leading to reduced viral shedding preventing lung function decline, and improving recovery and overall outcomes for patients. It was surprisingly found that treatment with the pharmaceutical combination of the invention provides a greater effect to favorably alter the mRNA expression of key proteins involved in the viral lifecycle and host cell response compared to treatment with a PPAR agonist or a p38 inhibitor alone. Moreover, the pharmaceutical combination was unexpectedly found to synergistically regulate the expression of genes implicated in severity of response to COVID-19 and to synergistically inhibit SARS-CoV-2 replication.

Accordingly, in a first aspect, the present invention provides a pharmaceutical combination comprising:
(a) a PPAR agonist;
(b) a p38 kinase inhibitor; and optionally
(c) one or more pharmaceutically acceptable diluents, excipients or carriers for use in a method of preventing or treating viral diseases or disorders in a subject.

In a further aspect, the present invention provides a kit for use in a method of preventing or treating viral diseases or disorders in a subject, comprising a pharmaceutical combination comprising:
(a) a PPAR agonist;
(b) a p38 kinase inhibitor; and optionally
(c) one or more pharmaceutically acceptable diluents, excipients or carriers; and instructions for using the kit.

### BRIEF DESCRIPTION OF THE FIGURES

FIG. 1: Pamapimod inhibits shedding of new viruses as determined by qRT-PCR into cell culture supernatants from Sars-CoV-2 infected cells. Left Panel: dose-response of antiviral activity. Right Panel: cell toxicity determined by WST assay. Remdesivir was used as positive control.

FIG. 2: Western blot analysis detection of SARS-CoV-2 nucleocapsid protein in cell supernatants treated with pamapimod vs. remdesivir.

FIG. 3: Inhibition of shedding of new viruses after treatment with different p38MAPK inhibitors as determined by qRT-PCR. Concentration-response of antiviral activity.

FIG. 4: Inhibition of shedding of new viruses after treatment with pioglitazone as determined by qRT-PCR. Concentration-response of antiviral activity.

FIG. 5: Inhibition of shedding of new viruses after treatment with combination of pamapimod and pioglitazone determined by qRT-PCR into culture supernatants.

FIG. 6: Inhibition of replication of the B1.1.7 and B1.351 SARS-CoV-2 variants by pamapimod as determined by qRT-PCR Concentration-response of antiviral activity.

### DETAILED DESCRIPTION OF THE INVENTION

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any embodiment. The invention extends to any novel one, or any novel combination, of the features disclosed in this specification (including any accompanying claims, abstract and drawings), or to any novel one, or any novel combination, of the steps of any method or process so disclosed.

The terms “comprising”, “having”, and “including” are to be construed as open-ended terms (i.e., meaning “including, but not limited to”) unless otherwise noted.

The term “pharmaceutically acceptable diluents, excipients or carriers” as used herein refers to diluents, excipients or carriers that are suitable for use with humans and/or animals without undue adverse side effects (such as toxicity, irritation, and allergic response) commensurate with a reasonable benefit/risk ratio. “Diluents” are agents which are added to the bulk volume of the active agent making up the solid composition. As a result, the size of the solid composition increases, which makes it easier to handle. Diluents are convenient when the dose of drug per solid composition is low and the solid composition would otherwise be too small. “Excipients” can be binders, lubricants, glidants, coating additives or combinations thereof. Thus, excipients are intended to serve multiple purposes. “Carriers” can be solvents, suspending agents or vehicles, for delivering the instant compounds to a subject.

The term “viral diseases or disorders” is intended to refer to medical conditions of the body known in the art related to diseases or disorders caused by viral infection that leads to increased viral infectivity of others, and more severe morbidities in the infected patient. The term “viral diseases or disorders” also refers to a group of viral infections caused by viruses affecting the lung including SARS-CoV-2 (Coronavirus associated with COVID-19); SARS-CoV(Coronavirus associated with SARS); HCoV (human coronavirus); HA and NA influenza viruses; ADV (adenovirus); HBoV (human bocavirus); HMPV (human metapneumovirus); HPIV (human parainfluenza virus); HRSV (human respiratory syncytial virus); HRV (human rhinovirus).

The term “SARS-CoV-2” comprises the SARS-CoV-2 virus originated by zoonotic transmission to humans in Wuhan, China and variants thereof. Variants have emerged around the world and new variants will continue to be identified. SARS-CoV-2 variants are classified according to a scheme that defines three classes: i. Variant of Interest; ii. Variant of Concern; iii. Variant of High Consequence. The B.1.526, B.1.526.1, B.1.525, and P.2 variants are classified as variants of interest. The B.1.1.7, B.1.351, P.1, B.1.427, and B.1.429 variants are classified as variants of concern. The term “SARS-CoV-2” comprises in particular the SARS-CoV-2 virus and i. Variant of Interest; ii. Variant of Concern; and iii. Variant of High Consequence, more particular SARS-CoV-2, B1.1.7 SARS-CoV-2 and B1.351 SARS-CoV-2.

The term “pharmaceutically acceptable salt” of a compound means a salt that is pharmaceutically acceptable and that it possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxy-benzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-enel-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e. g. an alkaline metal ion, an alkaline earth metal ion, or an aluminum ion; or coordinates with an organic base such as ethanolamine, diethanolamine, triethanolamine, tromethamine, N-methylglucamine, and the like. Preferred salts comprise acid addition salts formed with hydrochloric acid.

The terms “subject” and “patient” are used herein interchangeably and refer to mammals, in particular humans.

The term “about” as used herein refers to +/−10% of a given measurement.

In a first aspect, the present invention provides a pharmaceutical combination comprising:
(a) a PPAR agonist;
(b) a p38 kinase inhibitor; and optionally
(c) one or more pharmaceutically acceptable diluents, excipients or carriers for use in a method of preventing or treating viral diseases or disorders in a subject.

PPAR Agonists

The term “PPAR agonist” as used herein refers to a drug that is activating peroxisome proliferator activated receptor (PPAR) such as PPAR gamma receptor, PPAR alpha receptor, PPAR delta receptor or combinations thereof and includes PPAR gamma agonists such as e.g. pioglitazone, troglitazone or rosiglitazone, PPAR alpha agonists such as e.g. fibrates such as bezafibrate, fenofibrate (fenofibric acid), clofibrate or gemfibrozil, PPAR dual agonists (PPAR alpha/gamma or PPAR alpha/delta agonists) such as e.g. aleglitazar, muraglitazar, tesaglitazar, ragaglitazar, saroglitazar, GFT505 or naveglitazar, PPAR delta agonists such as e.g. GW501516, PPAR pan agonists (PPAR alpha/delta/gamma agonists) or selective PPAR modulators such as e.g. INT131 and the pharmaceutically acceptable salts of these compounds. Usually PPAR gamma agonists, PPAR modulators, PPAR alpha agonists and/or PPAR alpha/gamma dual agonists are used in the pharmaceutical combinations of the present invention, in particular PPAR gamma agonists, PPAR alpha agonists and/or PPAR alpha/gamma dual agonists are used in the pharmaceutical combinations of the present invention, more particularly PPAR gamma agonists and/or PPAR alpha agonists selected from the group consisting of pioglitazone, rosiglitazone, troglitazone, fenofibrate, bezafibrate and pharmaceutically acceptable salts thereof, even more particularly PPAR gamma agonists selected from the group consisting of pioglitazone, rosiglitazone, troglitazone and pharmaceutically acceptable salts thereof, preferably pioglitazone or pharmaceutically acceptable salts thereof. PPAR alpha agonists used in the pharmaceutical combinations of the present invention are selected from the group consisting of bezafibrate, fenofibrate (fenofibric acid), clofibrate, gemfibrozil and pharmaceutically acceptable salts thereof, preferably bezafibrate, fenofibrate (fenofibric acid) or pharmaceutically acceptable salts thereof, more preferably bezafibrate or pharmaceutically acceptable salts thereof. PPAR alpha/gamma dual agonists used in the pharmaceutical combinations of the present invention are selected from the group consisting of aleglitazar, muraglitazar, tesaglitazar, ragaglitazar, saroglitazar, GFT505, naveglitazar and pharmaceutically acceptable salts thereof, preferably muraglitazar, tesaglitazar or pharmaceutically acceptable salts thereof. Preferably PPAR gamma agonists and/or PPAR alpha agonists are used in the pharmaceutical combinations of the present invention, more preferably PPAR gamma agonists or modulators and/or PPAR alpha agonists selected from the group consisting of pioglitazone, rosiglitazone, troglitazone, fenofibrate, bezafibrate, INT131 and pharmaceutically acceptable salts thereof, even more preferably PPAR gamma agonists selected from the group consisting of pioglitazone, rosiglitazone, troglitazone and pharmaceutically acceptable salts thereof are used. Even more preferably, pioglitazone or a pharmaceutically acceptable salt thereof, in particular pioglitazone hydrochloride is used in the pharmaceutical combinations of the present invention. In one embodiment, a thiazolidinedione PPAR agonist is used in the pharmaceutical combinations of the invention. Suitable thiazolidinedione PPAR agonists are for example pioglitazone, troglitazone, rosiglitazone or pharmaceutically acceptable salts thereof. A particularly suitable thiazolidinone PPAR agonist is pioglitazone or a pharmaceutically acceptable salt thereof, in particular pioglitazone hydrochloride.

Pioglitazone is described e.g. in U.S. Pat. No. 4,687,777 or in Dormandy J A, Charbonnel B, Eckland D J, Erdmann E, Massi-Benedetti M, Moules IK, Skene A M, Tan M H, Léfebvre PJ, Murray G D, Standl E, Wilcox R G, Wilhelmsen L, Betteridge J, Birkeland K, Golay A, Heine R J, Korinyi L, Laakso M, Mokán M, Norkus A, Pirags V, Podar T, Scheen A, Scherbaum W, Schernthaner G, Schmitz O, Skrha J, Smith U, Taton J; PROactive investigators. Lancet. 2005 Oct. 8; 366(9493):1279-89, and is represented by the structural formula indicated below:

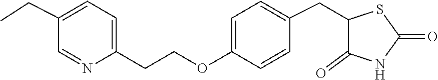 [SMILES: CCc1ccc(CCOc2ccc(CC3SC(=O)NC3=O)cc2)nc1]

Troglitazone is described e.g. in Florez J C, Jablonski K A, Sun M W, Bayley N, Kahn S E, Shamoon H, Hamman R F, Knowler W C, Nathan D M, Altshuler D; Diabetes Prevention Program Research Group. J Clin Endocrinol Metab. 2007 April; 92(4):1502-9 and is represented by the structural formula indicated below:

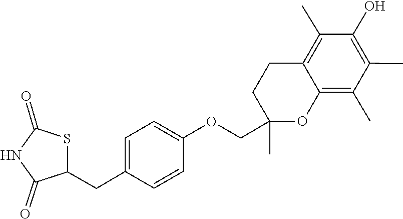 [SMILES: Cc1c(C)c2c(c(C)c1O)CCC(C)(COc1ccc(CC3SC(=O)NC3=O)cc1)O2]

Rosiglitazone is described e.g. in Nissen S E, Wolski K. N Engl J Med. 2007 Jun. 14; 356(24):2457-71. Erratum in: N Engl J Med. 2007 Jul. 5; 357(1):100. Fenofibrate is described e.g. in Bonds DE, Craven TE, Buse J, Crouse JR, Cuddihy R, Elam M, Ginsberg HN, Kirchner K, Marcovina S, Mychaleckyj JC, O'Connor P J, Sperl-Hillen J A. Diabetologia. 2012 June;55(6):1641-50 and is represented by the structural formula indicated below:

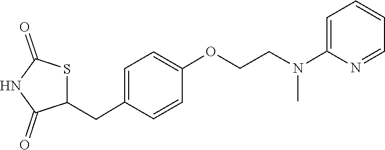 [SMILES: CN(CCOc1ccc(CC2SC(=O)NC2=O)cc1)c1ccccn1]

Bezafibrate is described e.g. in I. Goldenberg, M. Benderly, U. Goldbourt, Vascular health and risk management. 2008, 4(1): 131-141 and is represented by the structural formula indicated below:

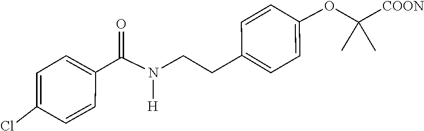

Clofibrate is described e.g. in Rabkin S W, Hayden M, Frohlich J. Atherosclerosis. 1988 October;73(2-3):233-40 and is represented by the structural formula indicated below:

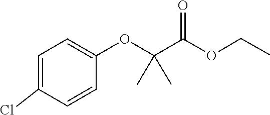 [SMILES: CCOC(=O)C(C)(C)Oc1ccc(Cl)cc1]

Fenofibrate (fenofibric acid) is described e.g. in Schima S M, Maciejewski S R, Hilleman DE, Williams M A, Mohiuddin S M. Expert Opin Pharmacother. 2010 April;11(5):731-8 and is represented by the structural formula indicated below:

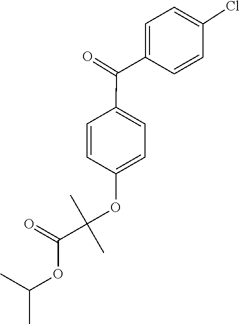 [SMILES: CC(C)OC(=O)C(C)(C)Oc1ccc(C(=O)c2ccc(Cl)cc2)cc1]

Gemfibrozil is described e.g. in Adabag A S, Mithani S, Al Aloul B, Collins D, Bertog S, Bloomfield H E; Veterans Affairs High-Density Lipoprotein Cholesterol Intervention Trial Study Group. Am Heart J. 2009 May; 157(5):913-8 and is represented by the structural formula indicated below:

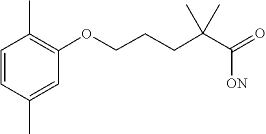

Aleglitazar is described e.g. in Lincoff A M, Tardif J C, Schwartz G G, Nicholls S J, Ryden L, Neal B, Malmberg K, Wedel H, Buse J B, Henry R R, Weichert A, Cannata R, Svensson A, Volz D, Grobbee D E; AleCardio Investigators. JAMA. 2014 Apr. 16; 311(15):1515-25 and is represented by the structural formula indicated below:

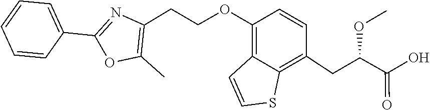 [SMILES: CO[C@@H](Cc1ccc(OCCc2nc(-c3ccccc3)oc2C)c2ccsc12)C(=O)O]

Muraglitazar is described e.g. in Fernandez M, Gastaldelli A, Triplitt C, Hardies J, Casolaro A, Petz R, Tantiwong P, Musi N, Cersosimo E, Ferrannini E, DeFronzo R A. Diabetes Obes Metab. 2011 October;13(10):893-902 and is represented by the structural formula indicated below:

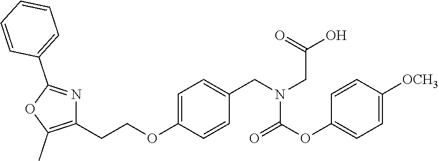 [SMILES: COc1ccc(OC(=O)N(CC(=O)O)Cc2ccc(OCCc3nc(-c4ccccc4)oc3C)cc2)cc1]

Tesaglitazar is described e.g. in Bays H, McElhattan J, Bryzinski B S; GALLANT 6 Study Group. Diab Vase Dis Res. 2007 September;4(3):181-93 and is represented by the structural formula indicated below:

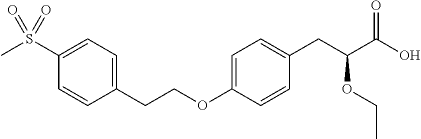 [SMILES: CCO[C@@H](Cc1ccc(OCCc2ccc(S(C)(=O)=O)cc2)cc1)C(=O)O]

Ragaglitazar is described e.g. in Saad M F, Greco S, Osei K, Lewin A J, Edwards C, Nunez M, Reinhardt R R; Ragaglitazar Dose-Ranging Study Group. Diabetes Care. 2004 June;27(6):1324-9 and is represented by the structural formula indicated below:

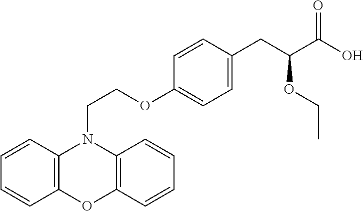 [SMILES: CCO[C@@H](Cc1ccc(OCCN2c3ccccc3Oc3ccccc32)cc1)C(=O)O]

Saroglitazar is described e.g. in Agrawal R. Curr Drug Targets. 2014 February;15(2):151-5. and is represented by the structural formula indicated below:

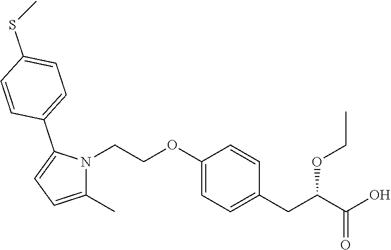 [SMILES: CCO[C@@H](Cc1ccc(OCCn2c(C)ccc2-c2ccc(SC)cc2)cc1)C(=O)O]

Naveglitazar is described e.g. in Ahlawat P, Srinivas N R. Eur J Drug Metab Pharmacokinet. 2008 July-September;33(3):187-90. GW501516 is described e.g. in Wang X, Sng M K, Foo S, Chong H C, Lee W L, Tang M B, Ng K W, Luo B, Choong C, Wong M T, Tong B M, Chiba S, Loo S C, Zhu P, Tan N S. J Control Release. 2015 Jan. 10; 197:138-47 and is represented by the structural formula indicated below:

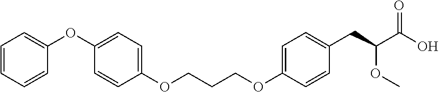 [SMILES: CO[C@@H](Cc1ccc(OCCCOc2ccc(Oc3ccccc3)cc2)cc1)C(=O)O]

GFT505 is described e.g. in Cariou B, Staels B. Expert Opin Investig Drugs. 2014 October;23(10):1441-8 and is represented by the structural formula indicated below:

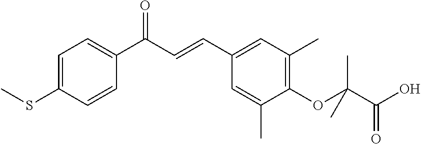 [SMILES: CSc1ccc(C(=O)/C=C/c2cc(C)c(OC(C)(C)C(=O)O)c(C)c2)cc1]

INT131 is described e.g. in. Taygerly J P, McGee L R, Rubenstein S M, Houze J B, Cushing T D, Li Y, Motani A, Chen J L, Frankmoelle W, Ye G, Learned M R, Jaen J, Miao S, Timmermans P B, Thoolen M, Kearney P, Flygare J, Beckmann H, Weiszmann J, Lindstrom M, Walker N, Liu J, Biermann D, Wang Z, Hagiwara A, Iida T, Aramaki H, Kitao Y, Shinkai H, Furukawa N, Nishiu J, Nakamura M. Bioorg Med Chem. 2013 Feb. 15; 21(4):979-92 and is represented by the structural formula indicated below:

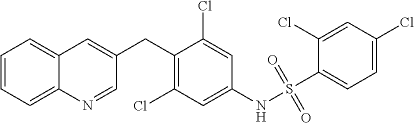 [SMILES: O=S(=O)(Nc1cc(Cl)c(Cc2cnc3ccccc3c2)c(Cl)c1)c1ccc(Cl)cc1Cl]

PPAR activation by the PPAR agonist is usually strong in the low nanomolar range to low micromolar range, e.g in a range of 0.1 nM to 100 μM. In some embodiments the PPAR activation is weak or partial, i.e. a PPAR agonist is used in the methods of the present invention which yields maximal activation of PPAR-receptor in a reporter assay system of 10% to 100% compared to a reference PPAR agonist which is known to causes a maximum PPAR activation.

p38 Kinase Inhibitors

The term “p38 kinase inhibitor” or “p38 inhibitor” which are both used interchangeably herein refers to a drug that is inhibiting a p38 mitogen-activated protein (MAP) kinase, such as p38-alpha (MAPK14), p38-beta (MAPK11), p38-gamma (MAPK12/ERK6), and/or p38-delta (MAPK13/SAPK4). Examples of p38 inhibitors include compounds of formulae I and II and pharmaceutically acceptable salts thereof as defined herein. Further examples of p38 inhibitors include pamapimod, acumapimod, losmapimod, dilmapimod, semapimod, AZD7624, ARRY-371797, LY2228820, R9111, PH-797804, BIRB 796, VX-702, VX-745, SB 239063, SB202190, SCIO 469, BMS 582949 and pharmaceutically acceptable salts thereof.

In one embodiment, the pharmaceutical combination according to the invention comprises:
(a) a PPAR agonist;
(b) a p38 kinase inhibitor; and optionally
(c) one or more pharmaceutically acceptable diluents, excipients or carriers; wherein said p38 inhibitor is inhibiting p38-alpha, p38-beta, p38-gamma or p38-delta or combinations thereof, preferably inhibiting p38-alpha and/or p38-beta, more preferably inhibiting p38-alpha.

In a further embodiment, the p38 inhibitor for use in a pharmaceutical combination according to the invention is a compound of formula I or II

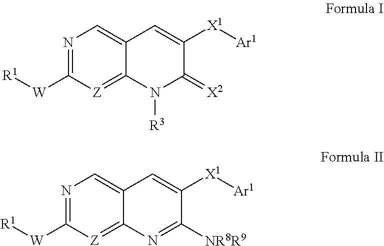

or a pharmaceutically acceptable salt thereof, wherein
Z is N or CH;
W is NR²
X¹ is O, NR⁴ (where R⁴ is hydrogen or alkyl), S, or CR⁵R⁶(where R⁵ and R⁶ are independently hydrogen or alkyl) or C═O;
X² is O or NR⁷;
Ar¹ is aryl or heteroaryl;
R² is hydrogen, alkyl, acyl, alkoxycarbonyl, aryloxycarbonyl, heteroalkylcarbonyl, heteroalkyloxycarbonyl or —R²¹—R²² where R²¹ is alkylene or —C(═O)— and R²² is alkyl or alkoxy;
R¹ is hydrogen, alkyl, haloalkyl, aryl, aralkyl, heteroaryl, heteroaralkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl, R¹²—SO₂-heterocycloamino (where R¹² is haloalkyl, aryl, aralkyl, heteroaryl or heteroaralkyl),—Y¹—C(O)—Y²-R¹¹ (where Y¹ and Y² are independently either absent or an alkylene group and R″ is hydrogen, alkyl, haloalkyl, hydroxy, alkoxy, amino, monoalkylamino or dialkylamino), (heterocyclyl)(cycloalkyl)alkyl or (heterocyclyl)(heteroaryl)alkyl;
R³ is hydrogen, alkyl, cycloalkyl, cycloalkylalkyl, aryl, aralkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino), amino, monoalkylamino, dialkylamino or NR³-Y³-R³³ (where Y³ is —C(O), —C(O)O—, —C(O)NR³⁴, S(O)₂ or S(O)₂NR⁵; R2, R³⁴ and R³⁵ are independently hydrogen or alkyl; and R³³ is hydrogen, alkyl, cycloalkyl, cycloalkylalkyl, heteroalkyl or optionally substituted phenyl) or acyl;
R⁷ is hydrogen or alkyl; and
R⁸ and R⁹ are independently hydrogen, alkyl, aryl, aralkyl, cycloalkyl, cycloalkylalkyl, heteroalkyl, alkylsulfonyl, arylsulfonyl, —C(O)—R⁸¹ (where R⁸¹ is alkyl, aryl, aralkyl, cycloalkyl, cycloalkylalkyl, heteroalkyl, alkoxy, aryloxy, amino, mono- or dialkylamino, arylamino or aryl(alkyl)amino) or R⁸ and R⁹ together form=CR⁸²R⁸³ (where R⁸² and R⁸³ are independently hydrogen, alkyl, cycloalkyl, cycloalkylalkyl or optionally substituted phenyl) and optionally one or more pharmaceutically acceptable diluents, excipients or carriers.

In a preferred embodiment, the p38 inhibitor for use in a pharmaceutical combination according to the invention is a compound of formula I

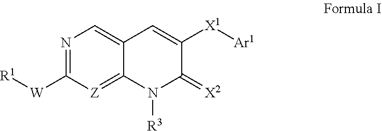

or a pharmaceutically acceptable salt thereof, wherein
Z is N or CH;
W is NR²
X¹ is O, NR⁴ (where R⁴ is hydrogen or alkyl), S, or CR⁵R⁶(where R⁵ and R⁶ are independently hydrogen or alkyl) or C═O;
X² is O or NR⁷;
Ar¹ is aryl or heteroaryl;
R² is hydrogen, alkyl, acyl, alkoxycarbonyl, aryloxycarbonyl, heteroalkylcarbonyl, heteroalkyloxycarbonyl or —R²—R² where R²¹ is alkylene or —C(═O)— and R²² is alkyl or alkoxy;
R¹ is hydrogen, alkyl, haloalkyl, aryl, aralkyl, heteroaryl, heteroaralkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl, R¹²—SO₂-heterocycloamino (where R¹² is haloalkyl, aryl, aralkyl, heteroaryl or heteroaralkyl),—Y¹—C(O)—Y²-R¹¹(where Y¹ and Y² are independently either absent or an alkylene group and R″ is hydrogen, alkyl, haloalkyl, hydroxy, alkoxy, amino, monoalkylamino or dialkylamino), (heterocyclyl)(cycloalkyl)alkyl or (heterocyclyl)(heteroaryl)alkyl;
R³ is hydrogen, alkyl, cycloalkyl, cycloalkylalkyl, aryl, aralkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino), amino, monoalkylamino, dialkylamino or NR³²-Y³-R³³ (where Y³ is —C(O), —C(O)O—, —C(O)NR³⁴, S(O)₂ or S(O)₂NR³⁵; R³², R³⁴ and R³⁵ are independently hydrogen or alkyl; and R³³ is hydrogen, alkyl, cycloalkyl, cycloalkylalkyl, heteroalkyl or optionally substituted phenyl) or acyl; and
R⁷ is hydrogen or alkyl
   and optionally one or more pharmaceutically acceptable diluents, excipients or carriers.

In a further embodiment, the p38 inhibitor for use in a pharmaceutical combination according to the invention is a compound of formula I wherein X¹ is NR⁴ and X² is NR⁷ or X¹ and X² are each O, wherein R⁴ and R⁷ are as defined above.

In a further embodiment, the p38 inhibitor for use in a pharmaceutical combination according to the invention is a compound of formula I wherein X¹ is NR⁴ or O and X² is NR⁷ or O, wherein R⁴ and R⁷ are as defined above.

In a further embodiment, the p38 inhibitor for use in a pharmaceutical combination according to the invention is a compound of formula I wherein W is NR² and wherein R² is hydrogen, alkyl, heteroalkyl, acyl or alkoxycarbonyl, preferably hydrogen or alkyl, more preferably hydrogen.

In yet a further embodiment, the p38 inhibitor for use in a pharmaceutical combination according to the invention is a compound of formula I wherein R¹ is hydrogen, alkyl, haloalkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl or (heterocyclyl)(cycloalkyl)alkyl.

In a preferred embodiment, the p38 inhibitor for use in a pharmaceutical combination according to the invention is a compound of formula I wherein R² is hydrogen and R¹ is heteroalkyl or vice versa.

In yet a further embodiment, the p38 inhibitor for use in a pharmaceutical combination according to the invention is a compound of formula I wherein R¹ is hydrogen, alkyl, haloalkyl, heteroalkyl or cyanoalkyl.

In yet a further embodiment, the p38 inhibitor for use in a pharmaceutical combination according to the invention is a compound of formula I wherein R¹ is cycloalkyl, cycloalkylalkyl, heteroalkylsubstituted cycloalkyl, heterosubstituted cycloalkyl, heterocyclyl, heterocyclylalkyl or (heterocyclyl)(cycloalkyl)alkyl.

In a preferred embodiment, the p38 inhibitor for use in a pharmaceutical combination according to the invention is a compound of formula I wherein each of R¹ and R² is independently selected from hydrogen and hydroxyalkyl, preferably from hydrogen, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl, 2-(hydroxymethyl)-3-hydroxypropyl, 3-hydroxy-1-(2-hydroxyethyl)-propyl and 2-hydroxy-1-methylethyl, more preferably from hydrogen, 2-hydroxyethyl, 2,3-dihydroxypropyl and 1-(hydroxymethyl)2-hydroxyethyl, most preferably from hydrogen, 2-hydroxy-propyl, 3-hydroxy-1-(2-hydroxyethyl)-propyl and 2-hydroxy-1-methylethyl.

In yet a further embodiment, the p38 inhibitor for use in a pharmaceutical combination according to the invention is a compound of formula I wherein R³ is hydrogen, alkyl, cycloalkyl, cycloalkylalkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino) or acyl.

In yet a further embodiment, the p38 inhibitor for use in a pharmaceutical combination according to the invention is a compound of formula I wherein R³ is hydrogen, alkyl, haloalkyl, heteroalkyl, cyanoalkyl, cycloalkyl or cycloalkylalkyl.

In yet a further embodiment, the p38 inhibitor for use in a pharmaceutical combination according to the invention is a compound of formula I wherein R³ is hydrogen, alkyl, haloalkyl, heteroalkyl or cyanoalkyl.

In yet a further embodiment, the p38 inhibitor for use in a pharmaceutical combination according to the invention is a compound of formula I wherein R³ is cycloalkyl or cycloalkylalkyl.

In yet a further embodiment, the p38 inhibitor for use in a pharmaceutical combination according to the invention is a compound of formula I wherein X¹ and X² are both O.

In yet a further embodiment, the p38 inhibitor for use in a pharmaceutical combination according to the invention is a compound of formula I wherein R¹ is alkyl or heteroalkyl.

In yet a further embodiment, the p38 inhibitor for use in a pharmaceutical combination according to the invention is a compound of formula I wherein R¹ is heteroalkyl, preferably 3-hydroxy-1-(2-hydroxyethyl)-propyl or 2-hydroxy-1-methylethyl.

In yet a further embodiment, the p38 inhibitor for use in a pharmaceutical combination according to the invention is a compound of formula I wherein R³ is alkyl or heteroalkyl.

In yet a further embodiment, the p38 inhibitor for use in a pharmaceutical combination according to the invention is a compound of formula I wherein R³ is alkyl, preferably C1-C5 alkyl, more preferably C1-C4 alkyl, more preferably C1-C3 alkyl. In a particularly preferred embodiment, R³ is ethyl or methyl, preferably methyl.

In yet a further embodiment, the p38 inhibitor for use in a pharmaceutical combination according to the invention is a compound of formula I wherein R³ is heteroalkyl, preferably 2-hydroxy-propyl.

In yet a further embodiment, the p38 inhibitor for use in a pharmaceutical combination according to the invention is a compound of formula I wherein W is NH.

In yet a further embodiment, the p38 inhibitor for use in a pharmaceutical combination according to the invention is a compound of formula I wherein Z is N.

In yet a further embodiment, the p38 inhibitor for use in a pharmaceutical combination according to the invention is a compound of formula I wherein Ar¹ is aryl, preferably phenyl optionally substituted with one, two or three halo substituents, most preferably phenyl substituted with two halo substituents in ortho and para position. In a particularly preferrred embodiment, Ar¹ is 2,4-difluorophenyl.

In yet a further embodiment, the p38 inhibitor for use in a pharmaceutical combination according to the invention is a compound of formula I wherein X¹ is NR⁴ and X² is NR⁷ or X and X² are each O, wherein R⁴ and R⁷ are as defined above; and wherein
R¹ is hydrogen, alkyl, haloalkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl or (heterocyclyl)(cycloalkyl)alkyl; and wherein
R³ is hydrogen, alkyl, cycloalkyl, cycloalkylalkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino) or acyl; and wherein
W is NR², wherein R² is hydrogen, alkyl, acyl or alkoxycarbonyl; and wherein
Ar¹ is aryl; and wherein
Z is N.

In a preferred embodiment, the p38 inhibitor for use in a pharmaceutical combination according to the invention is a compound of formula I wherein X¹ and X² are each O and wherein Z is N and wherein W is NH and wherein Ar¹ is phenyl optionally substituted by one, two or three halo substituents and wherein R¹ is heteroalkyl and wherein R³ is alkyl or heteroalkyl.

In a further embodiment, the p38 inhibitor for use in a pharmaceutical combination according to the invention is a compound of formula II

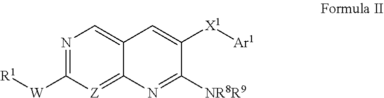

or a pharmaceutically acceptable salt thereof, wherein Ar¹, W, X¹, Z, R¹, R⁸ and R⁹ are as defined in any of the embodiments above.

Unless otherwise stated, the following terms have the meanings given below:

“Acyl” means a radical —C(O)R, where R is hydrogen, alkyl, cycloalkyl, cycloalkylalkyl, phenyl or phenylalkyl wherein alkyl, cycloalkyl, cycloalkylalkyl, and phenylalkyl are as defined herein. Representative examples include, but are not limited to formyl, acetyl, cylcohexylcarbonyl, cyclohexylmethylcarbonyl, benzoyl, benzylcarbonyl, and the like.

“Acylamino” means a radical-NR′C(O)R, where R′ is hydrogen or alkyl, and R is hydrogen, alkyl, cycloalkyl, cycloalkylalkyl, phenyl or phenylalkyl wherein alkyl, cycloalkyl, cycloalkylalkyl, and phenylalkyl are as defined herein. Representative examples include, but are not limited to formylamino, acetylamino, cylcohexylcarbonylamino, cyclohexylmethyl-carbonylamino, benzoylamino, benzylcarbonylamino, and the like.

“Alkoxy” means a radical —OR where R is an alkyl as defined herein. Examples are methoxy, ethoxy, propoxy, butoxy and the like.

“Alkoxycarbonyl” means a radical R—O—C(O)—, wherein R is an alkyl as defined herein.

“Alkyl” means a linear saturated monovalent hydrocarbon radical of one to six carbon atoms or a branched saturated monovalent hydrocarbon radical of three to six carbon atoms.

Examples include methyl, ethyl, propyl, 2-propyl, n-butyl, iso-butyl, tert-butyl, pentyl, and the like. Preferred are C1-C3 alkyl groups, in particular ethyl and methyl.

“Alkylsulfonyl” means a radical R—S(O)₂—, wherein R is alkyl as defined herein.

“Alkylene” means a linear saturated divalent hydrocarbon radical of one to six carbon atoms or a branched saturated divalent hydrocarbon radical of three to six carbon atoms. Examples are methylene, ethylene, 2,2-dimethylethylene, propylene, 2-methylpropylene, butylen, pentylene, and the like.

“Aryl” means a monovalent monocyclic or bicyclic aromatic hydrocarbon radical which is optionally substituted independently with one or more substituents, preferably one, two or three substituents preferably selected from the group consisting of alkyl, hydroxy, alkoxy, haloalkyl, haloalkoxy, Y—C(O)—R (where Y is absent or an alkylene group and R is hydrogen, alkyl, haloalkyl, haloalkoxy, hydroxy, alkoxy, amino, monoalkylamino or dialkylamino), heteroalkyl, heteroalkyloxy, heteroalkylamino, halo, nitro, cyano, amino, monoalkylamino, dialkylamino, alkylsulfonylamino, heteroalkylsulfonylamino, sulfonamido, methylenedioxy, ethylenedioxy, heterocyclyl or heterocyclylalkyl. Monocyclic aryl groups, optionally substituted as described above, are preferred. More specifically, the term aryl includes, but is not limited to, phenyl optionally substituted independently with one, two or three substituents preferably selected from the group consisting of alkyl, hydroxy, alkoxy, haloalkyl, haloalkoxy, Y—C(O)—R (where Y is absent or an alkylene group and R is hydrogen, alkyl, haloalkyl, haloalkoxy, hydroxy, alkoxy, amino, monoalkylamino or dialkylamino), heteroalkyl, heteroalkyloxy, heteroalkylamino, halo, nitro, cyano, amino, monoalkylamino, dialkylamino, alkylsulfonylamino, heteroalkylsulfonylamino, sulfonamido, methylenedioxy, ethylenedioxy, heterocyclyl and heterocyclylalkyl. Particularly preferred aryl groups are substituted phenyl groups selected from the group consisting of chlorophenyl, methoxyphenyl, 2-fluorophenyl, 2,4-difluorophenyl, 1-naphthyl and 2-naphthyl.

“Arylsulfonyl” means a radical R—S(O)₂—, wherein R is aryl as defined herein.

“Aralkyl” refers to an aryl group as defined herein bonded directly through an alkylene group, e.g. benzyl.

“Aryloxy” means a radical —OR where R is an aryl as defined herein, e. g. phenoxy.

“Aryloxycarbonyl” means a radical R—C(═O)— where R is aryloxy, e.g. phenoxycarbonyl.

“Cycloalkyl” refers to a saturated monovalent cyclic hydrocarbon radical of three to seven ring carbons or more specifically those of the specific compounds listed in the enclosed tables or being described in the examples. It is understood that these radicals can be grouped also in a group covering only such radicals but of the first or the second priority application or of both priority applications e. g., cyclopropyl, cyclobutyl, cyclohexyl, 4-methyl-cyclohexyl, and the like.

“Cycloalkylalkyl” means a radical —R^{a}R^{b} where R^{a} is an alkylene group and R^{b} is cycloalkyl group as defined herein, e. g., cyclohexylmethyl, and the like.

“Substituted cycloalkyl” means a cycloalkyl radical as defined herein with one, two or three (preferably one) ring hydrogen atoms independently replaced by cyano or —Y—C(O)R (where Y is absent or an alkylene group and R is hydrogen, alkyl, haloalkyl, hydroxy, alkoxy, amino, monoalkylamino, dialkylamino, or optionally substituted phenyl) or more specifically those of the specific compounds listed in the enclosed tables or being described in the examples.

“Halo” means fluoro, chloro, bromo, or iodo, preferably fluoro and chloro.

“Haloalkyl” means alkyl substituted with one or more same or different halo atoms, e. g. —CH₂C1, —CF₃, —CH₂CF₃, —CH₂CCl₃, and the like.

“Heteroalkyl” means an alkyl radical as defined herein wherein one, two or three hydrogen atoms have been replaced with a substituent independently selected from the group consisting of —OR^{a}, —N(O)ₙR^{b}R^{c}(where n is 0 or 1 if R^{b} and R^{c} are both independently alkyl, cycloalkyl or cycloalkylalkyl, and 0 if not) and —S(O)ₙR^{d} (where n is an integer from 0 to 2), with the understanding that the point of attachment of the heteroalkyl radical is through a carbon atom, wherein R^{a} is hydrogen, acyl, alkoxycarbonyl, alkyl, cycloalkyl, or cycloalkylalkyl; R^{b} and R^{c} are independently of each other hydrogen, acyl, alkoxycarbonyl, alkyl, cycloalkyl, cycloalkylalkyl, alkylsulfonyl, aminosulfonyl, mono- or dialkylaminosulfonyl, aminoalkyl, mono- or di-alkylaminoalkyl, hydroxyalkyl, alkoxyalkyl, hydroxyalkylsulfonyl or alkoxyalkylsulfonyl; and when n is 0, R^{d} is hydrogen, alkyl, cycloalkyl, cycloalkylalkyl or optionally substituted phenyl, and when n is 1 or 2, R^{d} is alkyl, cycloalkyl, cycloalkylalkyl, optionally substituted phenyl, amino, acylamino, monoalkylamino, or dialkylamino. Preferred heteroalkyl groups include hydroxyalkyl groups, preferably C1-C6 hydroxyalkyl groups. Representative examples include, but are not limited to, 2-hydroxyethyl, 2-hydroxy-propyl, 3-hydroxypropyl, 2-hydroxy-1-hydroxymethylethyl, 2-hydroxy-1-methylethyl, 2,3-dihydroxypropyl, 1-hydroxymethylethyl, 3-hydroxybutyl, 2,3-dihydroxybutyl, 2-hydroxy-1-methylpropyl, 3-hydroxy-1-(2-hydroxyethyl)-propyl, 2-aminoethyl, 3-aminopropyl, 2-methylsulfonylethyl, aminosulfonylmethyl, aminosulfonylethyl, aminosulfonylpropyl, methylaminosulfonylmethyl, methylaminosulfonylethyl, methylaminosulfonylpropyl, and the like. Particularly preferred heteroalkyl groups are 2-hydroxy-propyl, 3-hydroxy-1-(2-hydroxyethyl)-propyl or 2-hydroxy-1-methylethyl.

“Hydroxyalkyl” means an alkyl radical as defined herein, substituted with one or more, preferably one, two or three hydroxy groups, provided that the same carbon atom does not carry more than one hydroxy group. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl, 2-(hydroxymethyl)-3-hydroxypropyl, 3-hydroxy-1-(2-hydroxyethyl)-propyl and 2-hydroxy-1-methylethyl, preferably 2-hydroxyethyl, 2,3-dihydroxypropyl and 1-(hydroxymethyl)2-hydroxyethyl, more preferably 2-hydroxy-propyl, 3-hydroxy-1-(2-hydroxyethyl)-propyl and 2-hydroxy-1-methylethyl. Accordingly, as used herein, the term“hydroxyalkyl” is used to define a subset of heteroalkyl groups.

“Heteroalkylcarbonyl” means the group R^{a}—C(═O)—, where R^{a} is a heteroalkyl group.

Representative examples include acetyloxymethylcarbonyl, aminomethylcarbonyl, 4-acetyloxy-2,2-dimethyl-butan-2-oyl, 2-amino-4-methyl-pentan-2-oyl, and the like.

“Heteroalkyloxy” means the group R^{a}—O—, where R^{a} is a heteroalkyl group. Representative examples include (Me-C(═O)—O—CH₂—O—, and the like.

“Heteroalkyloxycarbonyl” means the group R^{a}—C(═O), where R^{a} is heteroalkyloxy. Representative examples include 1-acetyloxy-methoxycarbonyl (Me-C(═O)—OCH₂—O—C(═O)—) and the like.

“Heteroaryl” means a monovalent monocyclic or bicyclic radical of 5 to 12 ring atoms having at least one aromatic ring containing one, two, or three ring heteroatoms selected from the group consisting of N, O, or S, the remaining ring atoms being C, with the understanding that the attachment point of the heteroaryl radical will be on an aromatic ring. The heteroaryl ring is optionally substituted independently with one or more substituents, preferably one or two substituents, selected from the group consisting of alkyl, haloalkyl, heteroalkyl, hydroxy, alkoxy, halo, nitro or cyano. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl or benzothienyl, imidazo[1,2-a]-pyridinyl, imidazo[2,1-b]thiazolyl, and derivatives thereof.

“Heteroaralkyl” means a radical —R^{a}R^{b} where R^{a} is an alkylene group and R^{b} is a heteroaryl group, e. g. pyridin-3-ylmethyl, imidazolylethyl, pyridinylethyl, 3-(benzofuran-2-yl)propyl, and the like.

“Heteroalkylsubstituted cycloalkyl” means a cycloalkyl radical as defined herein wherein one, two or three hydrogen atoms in the cycloalkyl radical have been replaced with a heteroalkyl group with the understanding that the heteroalkyl radical is attached to the cycloalkyl radical via a carbon-carbon bond. Representative examples include, but are not limited to, 1-hydroxymethylcyclopentyl, 2-hydroxymethylcyclohexyl, and the like.

“Heterosubstituted cycloalkyl” means a cycloalkyl radical as defined herein wherein one, two or three hydrogen atoms in the cycloalkyl radical have been replaced with a substituent independently selected from the group consisting of hydroxy, alkoxy, amino, acylamino, monoalkylamino, dialkylamino, oxo(C═O), imino, hydroximino (═NOH), NR′SO₂R^{d} (where R′ is hydrogen or alkyl and R^{d} is alkyl, cycloalkyl, hydroxyalkyl, amino, monoalkylamino or dialkylamino), —X—Y—C(O)R (where X is O or NR′, Y is alkylene or absent, R is hydrogen, alkyl, haloalkyl, alkoxy, amino, monoalkylamino, dialkylamino, or optionally substituted phenyl, and R′ is H or alkyl), or —S(O)ₙR (where n is an integer from 0 to 2) such that when n is 0, R is hydrogen, alkyl, cycloalkyl, cycloalkylalkyl optionally substituted phenyl or thienyl, and when n is 1 or 2, R is alkyl, cycloalkyl, cycloalkylalkyl, optionally substituted phenyl, thienyl, amino, acylamino, monoalkylamino or dialkylamino. Representative examples include, but are not limited to, 2-, 3-, or 4-hydroxycyclohexyl, 2-, 3-, or 4-aminocyclohexyl, 2-, 3-, or 4-methanesulfonamido-cyclohexyl, and the like, preferably 4-hydroxycyclohexyl, 2-aminocyclohexyl or 4-methanesulfonamido-cyclohexyl.

“Heterosubstituted cycloalkyl-alkyl” means a radical R^{a}R^{b}—where R^{a} is a heterosubstituted cycloalkyl radical and R^{b} is an alkylene radical.

“Heterocycloamino” means a saturated monovalent cyclic group of 4 to 8 ring atoms, wherein one ring atom is N and the remaining ring atoms are C. Representative examples include piperidine and pyrrolidine.

“Heterocyclyl” means a saturated or unsaturated non-aromatic cyclic radical of 3 to 8 ring atoms in which one or two ring atoms are heteroatoms selected from N, O, or S(O)ₙ (where n is an integer from 0 to 2), the remaining ring atoms being C, where one or two C atoms may optionally be replaced by a carbonyl group. The heterocyclyl ring may be optionally substituted independently with one, two, or three substituents selected from the group consisting of alkyl, haloalkyl, heteroalkyl, halo, nitro, cyano, cyanoalkyl, hydroxy, alkoxy, amino, monoalkylamino, dialkylamino, aralkyl, —(X)ₙ—C(O)R (where X is O or NR′, n is 0 or 1, R is hydrogen, alkyl, haloalkyl, hydroxy (when n is 0), alkoxy, amino, monoalkylamino, dialkylamino, or optionally substituted phenyl, and R′ is H or alkyl), -alkylene-C(O)R^{a} (where R^{a} is alkyl, OR or NR′R″ and R is hydrogen, alkyl or haloalkyl, and R′ and R″ are independently hydrogen or alkyl), or —S(O)ₙR (where n is an integer from 0 to 2) such that when n is 0, R is hydrogen, alkyl, cycloalkyl, or cycloalkylalkyl, and when n is 1 or 2, R is alkyl, cycloalkyl, cycloalkylalkyl, amino, acylamino, monoalkylamino, dialkylamino or heteroalkyl. More specifically the term heterocyclyl includes, but is not limited to, tetrahydropyranyl, piperidino, N-methylpiperidin-3-yl, piperazino, N-methylpyrrolidin-3-yl, 3-pyrrolidino, morpholino, thiomorpholino, thiomorpholino-1-oxide, thiomorpholino-1,1-dioxide, 4-(1,1-dioxo-tetrahydro-2H-thiopyranyl), pyrrolinyl, imidazolinyl, N-methanesulfonyl-piperidin-4-yl, and the derivatives thereof.

“Heterocyclylalkyl” means a radical —R^{a}R^{b} where R^{a} is an alkylene group and R^{b} is a heterocyclyl group as defined above, e. g. tetrahydropyran-2-ylmethyl, 2- or 3-piperidinylmethyl, 3-(4-methyl-piperazin-1-yl)propyl and the like.

“(Heterocyclyl)(cycloalkyl)alkyl” means an alkyl radical wherein two hydrogen atoms have been replaced with a heterocyclyl group and a cycloalkyl group.

“(Heterocyclyl)(heteroaryl)alkyl” means an alkyl radical wherein two hydrogen atoms have been replaced with a heterocycyl group and a heteroaryl group.

“Amino” means a radical —NH₂.

“Monoalkylamino” means a radical —NHR where R is an alkyl, hydroxyalkyl, cycloalkyl, or cycloalkylalkyl group as defined above, e. g. methylamino, (1-methylethyl) amino, hydroxymethylamino, cyclohexylamino, cyclohexylmethylamino, cyclohexylethylamino, and the like.

“Dialkylamino” means a radical —NRR′ where R and R′ independently represent an alkyl, hydroxyalkyl, cycloalkyl, or cycloalkylalkyl group as defined herein. Representative examples include, but are not limited to dimethylamino, methylethylamino, di(1-methylethyl)amino, (methyl)(hydroxymethyl)amino, (cyclohexyl)(methyl)amino, (cyclohexyl)(ethyl)amino, (cyclohexyl)(propyl)amino, (cyclohexylmethyl)(methyl)amino, (cyclohexylmethyl)(ethyl)amino, and the like.

“Optionally substituted phenyl” means a phenyl ring which is optionally substituted independently with one or more substituents, preferably one, two or three substituents, more preferably two substituents selected from the group consisting of alkyl, hydroxy, alkoxy, haloalkyl, haloalkoxy, heteroalkyl, halo, nitro, cyano, amino, methylenedioxy, ethylenedioxy, and acyl, preferably halo, most preferably fluoro.

Thus, in one embodiment, the p38 inhibitor for use in a pharmaceutical combination according to the invention is selected from the group consisting of a compound of formula I or a pharmaceutically acceptable salt thereof, a compound of formula II or a pharmaceutically acceptable salt thereof, acumapimod, losmapimod, semapimod, AZD7624, ARRY-371797, LY2228820, R⁹¹¹¹, PH-797804, BIRB 796, VX-702, VX-745, SB 239063, SB202190, SCIO 469, and BMS 582949, and a pharmaceutically acceptable salt thereof, in particular selected from the group consisting of a compound of formula I or a pharmaceutically acceptable salt thereof, losmapimod, semapimod, AZD7624, ARRY-371797, LY2228820, R⁹¹¹¹, PH-797804, BIRB 796, VX-702, VX-745, SB 239063, SB202190, SCIO 469, and BMS 582949, and a pharmaceutically acceptable salt thereof.

Thus, in a preferred embodiment, the p38 inhibitor for use in a pharmaceutical combination according to the invention is selected from the group consisting of pamapimod, acumapimod, losmapimod, dilmapimod, semapimod, AZD7624, ARRY-371797, LY2228820, R⁹¹¹¹, PH-797804, BIRB 796, VX-702, VX-745, SB 239063, SB202190, SCIO 469, and BMS 582949, and a pharmaceutically acceptable salt thereof.

More preferred is a p38 inhibitor for use in a pharmaceutical combination according to the invention selected from the group consisting of pamapimod, losmapimod, LY2228820, BMS 582949, or pharmaceutically acceptable salts and mixtures thereof or selected from the group consisting of pamapimod, losmapimod, LY2228820, BMS 582949, or pharmaceutically acceptable salts thereof, or selected from the group consisting of pamapimod, losmapimod, dilmapimod, R⁹¹¹¹, LY2228820, BMS 582949, or pharmaceutically acceptable salts thereof, or selected from the group consisting of pamapimod, losmapimod, dilmapimod and R⁹¹¹¹, or pharmaceutically acceptable salts thereof, or selected from the group consisting of pamapimod, losmapimod, dilmapimod, ARRY-371797 and R⁹¹¹¹, or pharmaceutically acceptable salts thereof, more preferably pamapimod, losmapimod, or dilmapimod, or pharmaceutically acceptable salts thereof, even more preferably pamapimod or dilmapimod, or pharmaceutically acceptable salts thereof, in particular pamapimod, losmapimod, ARRY-371797, or pharmaceutically acceptable salts thereof, more particular pamapimod or a pharmaceutically acceptable salt thereof.

In a particularly preferred embodiment, the p38 inhibitor is pamapimod, having the chemical name 6-(2,4-Difluorophenoxy)-2-[3-hydroxy-1-(2-hydroxyethyl)-propylamino]-8-methyl-8H-pyrido[2,3-d]pyrimidin-7-one and the chemical formula III or a pharmaceutically acceptable salt thereof.

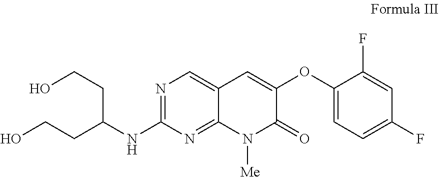 [SMILES: Cn1c(=O)c(Oc2ccc(F)cc2F)cc2cnc(NC(CCO)CCO)nc21]

Pamapimod and its synthesis are described e.g. in WO2008/151992 and in WO2002/064594 and in e.g. Hill R J, Dabbagh K, Phippard D, Li C, Suttmann R T, Welch M, Papp E, Song K W, Chang K C, Leaffer D, Kim Y-N, Roberts R T, Zabka T S, Aud D, Dal Porto J, Manning A M, Peng S L, Goldstein D M, and Wong B R; Pamapimod, a Novel p38 Mitogen-Activated Protein Kinase Inhibitor: Preclinical Analysis of Efficacy and Selectivity J Pharmacol Exp Ther. December 2008 327:610-619.

A further particularly preferred p38 inhibitor is losmapimod, having the chemical name 6-(5-((cyclopropylamino)carbonyl)-3-fluoro-2-methylphenyl)-N-(2,2-dimethylpropyl)-3-pyridinecarboxamide and the chemical formula IV or a pharmaceutically acceptable salt thereof.

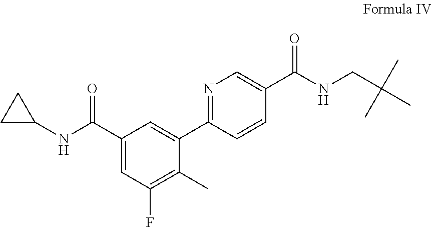 [SMILES: Cc1c(F)cc(C(=O)NC2CC2)cc1-c1ccc(C(=O)NCC(C)(C)C)cn1]

Losmapimod is described in e.g. Cheriyan J, Webb A J, Sarov-Blat L, Elkhawad M, Wallace S M, Mäki-Petäjä K M, Collier D J, Morgan J, Fang Z, Willette R N, Lepore J J, Cockcroft J R, Sprecher D L, Wilkinson I B. Inhibition of p38 mitogen-activated protein kinase improves nitric oxide-mediated vasodilatation and reduces inflammation in hypercholesterolemia. Circulation, 2011 February 8;123(5):515-23.

Yet a further particularly preferred p38 inhibitor is LY2228820, having the chemical name 3-(2,2-Dimethylpropyl)-5-[4-(4-fluorophenyl)-2-(2-methyl-2-propanyl)-1H-imidazol-5-yl]-3H-imidazo[4,5-b]pyridin-2-amine and the chemical formula V or a pharmaceutically acceptable salt thereof.

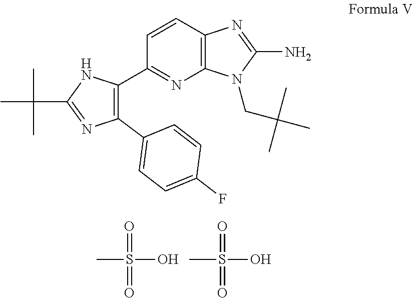

LY2228820 is described in e.g. Campbell R M, Anderson B D, Brooks N A, Brooks H B, Chan E M, De Dios A, Gilmour R, Graff J R, Jambrina E, Mader M, McCann D, Na S, Parsons S H, Pratt S E, Shih C, Stancato L F, Starling J J, Tate C, Velasco J A, Wang Y, Ye X S.

Characterization of LY2228820 dimesylate, a potent and selective inhibitor of p38 MAPK with antitumor activity. Mol Cancer Ther. 2014 February;13(2):364-74.

Yet a further particularly preferred p38 inhibitor is BMS 582949, having the chemical name 4-(5-(cyclopropylcarbamoyl)-2-methylphenylamino)-5-methyl-N-propylpyrrolo[1,2-f][1,2,4]triazine-6-carboxamide and the chemical formula VI or a pharmaceutically acceptable salt thereof

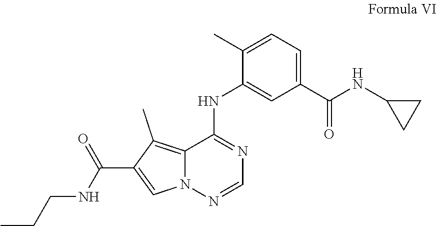 [SMILES: CCCNC(=O)c1cn2ncnc(Nc3cc(C(=O)NC4CC4)ccc3C)c2c1C]

BMS 582949 is described in e.g. Liu C, Lin J, Wrobleski S T, Lin S, Hynes J, Wu H, Dyckman A J, Li T, Wityak J, Gillooly K M, Pitt S, Shen D R, Zhang R F, McIntyre K W, Salter-Cid L, Shuster D J, Zhang H, Marathe P H, Doweyko A M, Sack J S, Kiefer S E, Kish K F, Newitt J A, McKinnon M, Dodd J H, Barrish J C, Schieven G L, Leftheris K. Discovery of 4-(5-(cyclopropylcarbamoyl)-2-methylphenylamino)-5-methyl-N-propylpyrrolo[1,2-f][1,2,4]triazine-6-carboxamide (BMS-582949), a clinical p38-alpha MAP kinase inhibitor for the treatment of inflammatory diseases. J Med Chem. 2010 Sep. 23; 53(18):6629-39.

Acumapimod has the chemical name 3-[5-Amino-4-(3-cyanobenzoyl)-1H-pyrazol-1-yl]-N-cyclopropyl-4-methylbenzamide and is described in e.g De Buck S, Hueber W, Vitaliti A, Straube F, Emotte C, Bruin G, Woessner R. Population P K-PD Model for Tolerance Evaluation to the p38 MAP Kinase Inhibitor BCT197. CPT Pharmacometrics Syst Pharmacol. 2015 December;4(12):691-700, and is represented by the structural formula indicated below:

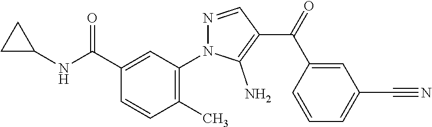 [SMILES: Cc1ccc(C(=O)NC2CC2)cc1-n1ncc(C(=O)c2cccc(C#N)c2)c1N]

Dilmapimod is described in e.g. Christie J D, Vaslef S, Chang P K, May A K, Gunn S R, Yang S, Hardes K, Kahl L, Powley W M, Lipson D A, Bayliffe Al, Lazaar A L. A Randomized Dose-Escalation Study of the Safety and Anti-Inflammatory Activity of the p38 Mitogen-Activated Protein Kinase Inhibitor Dilmapimod in Severe Trauma Subjects at Risk for Acute Respiratory Distress Syndrome. Crit Care Med. 2015 September;43(9):1859-69, and is represented by the structural formula indicated below:

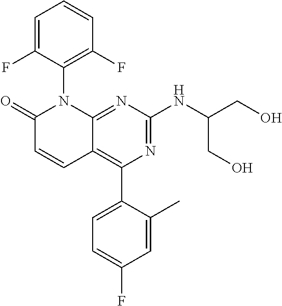 [SMILES: Cc1cc(F)ccc1-c1nc(NC(CO)CO)nc2c1ccc(=O)n2-c1c(F)cccc1F]

Semapimod is described in e.g. Bianchi, M.; Ulrich, P.; Bloom, O.; Meistrell m, M., I. I.; Zimmerman, G. A.; Schmidtmayerova, H.; Bukrinsky, M.; Donnelley, T.; Bucala, R.; Sherry, B.; Manogue, K. R.; Tortolani, A. J.; Cerami, A.; Tracey, K. J. (March 1995). Molecular Medicine (Cambridge, Mass.). 1 (3): 254-266 or in e.g. Wang J, Grishin A V, Ford H R. Experimental Anti-Inflammatory Drug Semapimod Inhibits TLR Signaling by Targeting the TLR Chaperone gp96. J Immunol. 2016 Jun. 15; 196(12):5130-7 and is represented by the structural formula as indicated below:

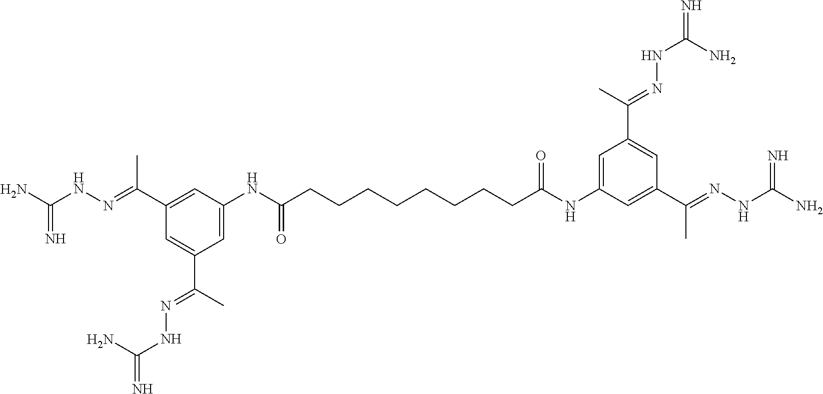 [SMILES: C/C(=N\NC(=N)N)c1cc(NC(=O)CCCCCCCCC(=O)Nc2cc(/C(C)=N/NC(=N)N)cc(/C(C)=N/NC(=N)N)c2)cc(/C(C)=N/NC(=N)N)c1]

AZD7624 is described in e.g. Patel N, Cunoosamy D, Hegelund-Myrback T, Pehrson R, Taib Z, Jansson P, Lundin S, Greenaway S, Clarke G, Siew L. AZD7624, an inhaled p38 inhibitor for COPD, attenuates lung and systemic inflammation after LPS Challenge in humans. Eur Resp J. DOI: 10.1183/13993003.1 September 2015, and is represented by the structural formula as indicated below:

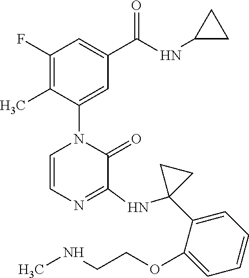 [SMILES: CNCCOc1ccccc1C1(Nc2nccn(-c3cc(C(=O)NC4CC4)cc(F)c3C)c2=O)CC1]

ARRY-371797 (also designated as ARRY-797 herein) is described in e.g. Muchir A, Wu W, Choi J C, Iwata S, Morrow J, Homma S, Worman H J. Abnormal p38-alpha mitogen-activated protein kinase signaling in dilated cardiomyopathy caused by lamin A/C gene mutation. Hum Mol Genet. 2012 Oct. 1; 21(19):4325-33, and is represented by the structural formula as indicated below:

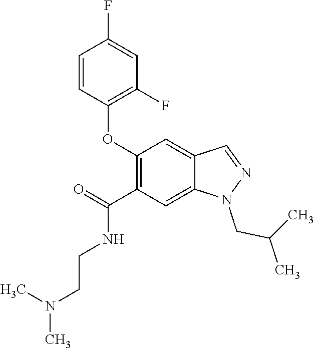

R⁹¹¹¹ and its synthesis is described in WO2005/047284 and in e.g. Hill R J, Dabbagh K, Phippard D, Li C, Suttmann R T, Welch M, Papp E, Song K W, Chang K C, Leaffer D, Kim Y-N, Roberts R T, Zabka T S, Aud D, Dal Porto J, Manning A M, Peng S L, Goldstein D M, and Wong B R; Pamapimod, a Novel p38 Mitogen-Activated Protein Kinase Inhibitor: Preclinical Analysis of Efficacy and Selectivity J Pharmacol Exp Ther. December 2008 327:610-619 and is represented by the structural formula as indicated below:

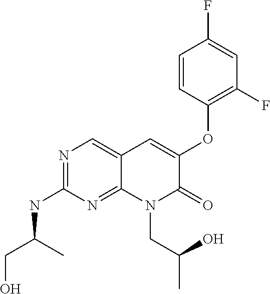 [SMILES: C[C@H](O)Cn1c(=O)c(Oc2ccc(F)cc2F)cc2cnc(N[C@@H](C)CO)nc21]

PH-797804 is described in e.g. Xing L, Devadas B, Devraj R V, Selness S R, Shieh H, Walker J K, Mao M, Messing D, Samas B, Yang J Z, Anderson G D, Webb E G, Monahan J B. Discovery and characterization of atropisomer PH-797804, a p38 MAP kinase inhibitor, as a clinical drug candidate. ChemMedChem. 2012 Feb. 6; 7(2):273-80, and is represented by the structural formula indicated below:

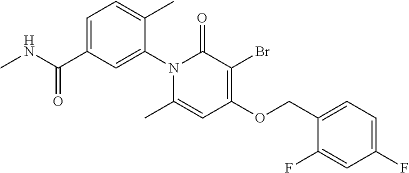 [SMILES: CNC(=O)c1ccc(C)c(-n2c(C)cc(OCc3ccc(F)cc3F)c(Br)c2=O)c1]

BIRB 796 is described in e.g. Dietrich J, Hulme C, Hurley L H. The design, synthesis, and evaluation of 8 hybrid DFG-out allosteric kinase inhibitors: a structural analysis of the binding interactions of Gleevec, Nexavar, and BIRB-796. Bioorg Med Chem. 2010 Aug. 1; 18(15):5738-48, and is represented by the structural formula indicated below:

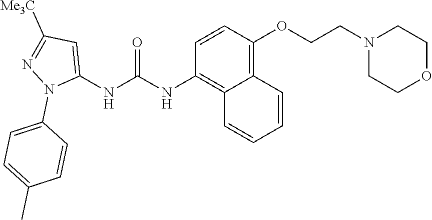

VX-702 is described in e.g. Damjanov N, Kauffman R S, Spencer-Green G T. Efficacy, pharmacodynamics, and safety of VX-702, a novel p38 MAPK inhibitor, in rheumatoid arthritis: results of two randomized, double-blind, placebo-controlled clinical studies. Arthritis Rheum. 2009 May; 60(5):1232-41, and is represented by the structural formula indicated below:

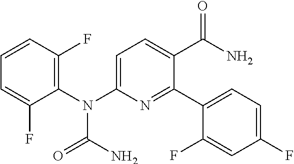 [SMILES: NC(=O)c1ccc(N(C(N)=O)c2c(F)cccc2F)nc1-c1ccc(F)cc1F]

VX-745 is described in e.g. Duffy J P, Harrington E M, Salituro F G, Cochran J E, Green J, Gao H, Bemis G W, Evindar G, Galullo V P, Ford P J, Germann U A, Wilson K P, Bellon S F, Chen G, Taslimi P, Jones P, Huang C, Pazhanisamy S, Wang Y M, Murcko M A, Su M S. The Discovery of VX-745: A Novel and Selective p38-alpha Kinase Inhibitor. ACS Med Chem Lett. 2011 Jul. 28; 2(10):758-63, and is represented by the structural formula indicated below:

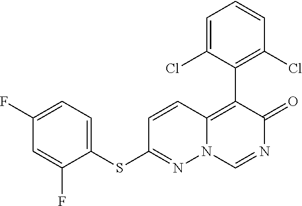 [SMILES: O=c1ncn2nc(Sc3ccc(F)cc3F)ccc2c1-c1c(Cl)cccc1Cl]

SB239063 is described in e.g. Strassburger M, Braun H, Reymann K G. Anti-inflammatory treatment with the p38 mitogen-activated protein kinase inhibitor SB239063 is neuroprotective, decreases the number of activated microglia and facilitates neurogenesis in oxygen-glucose-deprived hippocampal slice cultures. Eur J Pharmacol. 2008 Sep. 11; 592(1-3):55-61, and is represented by the structural formula indicated below:

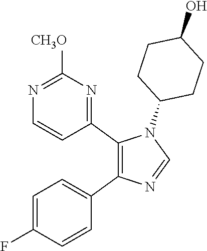

SB202190 is described in e.g. Hirosawa M, Nakahara M, Otosaka R, Imoto A, Okazaki T, Takahashi S. The p38 pathway inhibitor SB202190 activates MEK/MAPK to stimulate the growth of leukemia cells. Leuk Res. 2009 May; 33(5):693-9, and is represented by the structural formula indicated below:

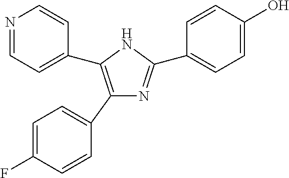 [SMILES: Oc1ccc(-c2nc(-c3ccc(F)cc3)c(-c3ccncc3)[nH]2)cc1]

SCI0469 is described in e.g. Sokol L, Cripe L, Kantarjian H, Sekeres M A, Parmar S, Greenberg P, Goldberg S L, Bhushan V, Shammo J, Hohl R, Verma A, Garcia-Manero G, Li Y P, Lowe A, Zhu J, List A F. Randomized, dose-escalation study of the p38-alpha MAPK inhibitor SCIO-469 in patients with myelodysplastic syndrome. Leukemia. 2013 April;27(4):977-80, and is represented by the structural formula indicated below:

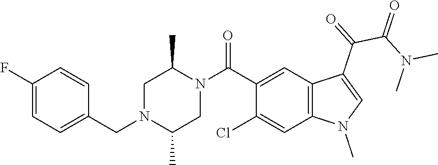 [SMILES: C[C@@H]1CN(Cc2ccc(F)cc2)[C@@H](C)CN1C(=O)c1cc2c(C(=O)C(=O)N(C)C)cn(C)c2cc1Cl]

In a further aspect, the present invention provides a p38 kinase inhibitor; and optionally one or more pharmaceutically acceptable diluents, excipients or carriers, for use in a method of preventing or treating viral diseases or disorders in a subject, preferably for use in a method of preventing or treating COVID-19 in a subject.

Useful p38 kinase inhibitors are as defined above. In a preferred embodiment, said p38 kinase inhibitors are inhibiting p38-alpha, p38-beta, p38-gamma or p38-delta or combinations thereof, preferably inhibiting p38-alpha and/or p38-beta, more preferably inhibiting p38-alpha. Further useful p38 kinase inhibitors are compunds of the formula I or II, or pharmaceutically acceptable salts thereof, as defined supra. Further useful p38 kinase inhibitors are compounds selected from the group consisting of a compound of formula I or a pharmaceutically acceptable salt thereof, a compound of formula II or a pharmaceutically acceptable salt thereof, acumapimod, losmapimod, semapimod, AZD7624, ARRY-371797, LY2228820, R⁹¹¹¹, PH-797804, BIRB 796, VX-702, VX-745, SB 239063, SB202190, SCIO 469, and BMS 582949, and a pharmaceutically acceptable salt thereof, in particular compounds selected from the group consisting of a compound of formula I or a pharmaceutically acceptable salt thereof, losmapimod, semapimod, AZD7624, ARRY-371797, LY2228820, R⁹¹¹¹, PH-797804, BIRB 796, VX-702, VX-745, SB 239063, SB202190, SCIO 469, and BMS 582949, and a pharmaceutically acceptable salt thereof. Further useful p38 kinase inhibitors are p38 kinase inhibitors selected from the group consisting of pamapimod, acumapimod, losmapimod, dilmapimod, semapimod, AZD7624, ARRY-371797, LY2228820, R⁹¹¹¹, PH-797804, BIRB 796, VX-702, VX-745, SB 239063, SB202190, SCIO 469, BMS 582949, and pharmaceutically acceptable salts thereof, in particular pamapimod, losmapimod, LY2228820, BMS 582949 or pharmaceutically acceptable salts and mixtures thereof, or selected from the group consisting of pamapimod, acumapimod, losmapimod, dilmapimod, semapimod, AZD7624, ARRY-371797, LY2228820, R⁹¹¹¹, PH-797804, BIRB 796, VX-702, VX-745, SB 239063, SB202190, SCIO 469, BMS 582949, and pharmaceutically acceptable salts thereof, in particular pamapimod, losmapimod, LY2228820, BMS 582949 or pharmaceutically acceptable salts thereof, or selected from the group consisting of pamapimod, losmapimod, dilmapimod, ARRY-371797 and R⁹¹¹¹, or pharmaceutically acceptable salts thereof, preferably pamapimod, losmapimod, dilmapimod or R⁹¹¹¹ or pharmaceutically acceptable salts thereof, more preferably pamapimod or dilmapimod or pharmaceutically acceptable salts thereof, in particular pamapimod, losmapimod, ARRY-371797, or pharmaceutically acceptable salts thereof, more particular pamapimod or a pharmaceutically acceptable salt thereof.

Pharmaceutical Combinations

As outlined above, in a first aspect, the present invention provides a pharmaceutical combination comprising:
(a) a PPAR agonist;
(b) a p38 kinase inhibitor; and optionally
(c) one or more pharmaceutically acceptable diluents, excipients or carriers for use in a method of preventing or treating viral diseases or disorders in a subject.

Useful PPAR agonists are as defined above. In one embodiment, said PPAR agonist is activating PPAR gamma and/or PPAR alpha. In a preferred embodiment, said PPAR agonist is selected from the group consisting of pioglitazone, rosiglitazone, troglitazone, fenofibrate, bezafibrate and pharmaceutically acceptable salts thereof. In a more preferred embodiment, said PPAR agonist is a PPAR gamma agonist, in particular pioglitazone or a pharmaceutically acceptable salt thereof. In a particularly preferred embodiment, said PPAR agonist is pioglitazone hydrochloride.

Useful p38 kinase inhibitors are as defined above. In a preferred embodiment, said p38 kinase inhibitors are inhibiting p38-alpha, p38-beta, p38-gamma or p38-delta or combinations thereof, preferably inhibiting p38-alpha and/or p38-beta, more preferably inhibiting p38-alpha. Further useful p38 kinase inhibitors are compunds of the formula I or II, or pharmaceutically acceptable salts thereof, as defined supra.

Further useful p38 kinase inhibitors are compounds selected from the group consisting of a compound of formula I or a pharmaceutically acceptable salt thereof, a compound of formula II or a pharmaceutically acceptable salt thereof, acumapimod, losmapimod, semapimod, AZD7624, ARRY-371797, LY2228820, R⁹¹¹¹, PH-797804, BIRB 796, VX-702, VX-745, SB 239063, SB202190, SCIO 469, and BMS 582949, and a pharmaceutically acceptable salt thereof, in particular compounds selected from the group consisting of a compound of formula I or a pharmaceutically acceptable salt thereof, losmapimod, semapimod, AZD7624, ARRY-371797, LY2228820, R⁹¹¹¹, PH-797804, BIRB 796, VX-702, VX-745, SB 239063, SB202190, SCIO 469, and BMS 582949, and a pharmaceutically acceptable salt thereof.

Further useful p38 kinase inhibitors are p38 kinase inhibitors selected from the group consisting of pamapimod, acumapimod, losmapimod, dilmapimod, semapimod, AZD7624, ARRY-371797, LY2228820, R⁹¹¹¹, PH-797804, BIRB 796, VX-702, VX-745, SB 239063, SB202190, SCIO 469, BMS 582949, and pharmaceutically acceptable salts thereof, in particular pamapimod, losmapimod, LY2228820, BMS 582949 or pharmaceutically acceptable salts and mixtures thereof, or selected from the group consisting of pamapimod, acumapimod, losmapimod, dilmapimod, semapimod, AZD7624, ARRY-371797, LY2228820, R⁹¹¹¹, PH-797804, BIRB 796, VX-702, VX-745, SB 239063, SB202190, SCIO 469, BMS 582949, and pharmaceutically acceptable salts thereof, in particular pamapimod, losmapimod, LY2228820, BMS 582949 or pharmaceutically acceptable salts thereof, or selected from the group consisting of pamapimod, losmapimod, dilmapimod, ARRY-371797 and R⁹¹¹¹, or pharmaceutically acceptable salts thereof, preferably pamapimod, losmapimod, dilmapimod or R⁹¹¹¹ or pharmaceutically acceptable salts thereof, more preferably pamapimod or dilmapimod or pharmaceutically acceptable salts thereof, in particular pamapimod, losmapimod, ARRY-371797, or pharmaceutically acceptable salts thereof, more particular pamapimod or a pharmaceutically acceptable salt thereof.

A pharmaceutical combination according to the invention is for example a combined preparation or a pharmaceutical composition, for simultaneous, separate or sequential use. The term “combined preparation” as used herein defines especially a “kit of parts” in the sense that said PPAR agonist and said p38 inhibitor can be dosed independently, either in separate form e.g. as separate tablets or by use of different fixed combinations with distinguished amounts of the active ingredients. The ratio of the amount of PPAR agonist to the amount of p38 inhibitor to be administered in the combined preparation can be varied, e.g. in order to cope with the needs of a patient sub-population to be treated or the needs of a single patient, which needs can be different due to age, sex, body weight, etc. of a patient. The individual parts of the combined preparation (kit of parts) can be administered simultaneously or sequentially, i.e. chronologically staggered, e.g. at different time points and with equal or different time intervals for any part of the kit of parts.

The term “pharmaceutical composition” refers to a fixed-dose combination (FDC) that includes the PPAR agonist and the p38 inhibitor combined in a single dosage form, having a predetermined combination of respective dosages.

The pharmaceutical combination further may be used as add-on therapy. As used herein, “add-on” or “add-on therapy” means an assemblage of reagents for use in therapy, the subject receiving the therapy begins a first treatment regimen of one or more reagents prior to beginning a second treatment regimen of one or more different reagents in addition to the first treatment regimen, so that not all of the reagents used in the therapy are started at the same time. For example, adding p38 inhibitor therapy to a patient already receiving PPAR agonist therapy and vice versa.

In a preferred embodiment, the pharmaceutical combination according to the invention is a combined preparation.

In a further preferred embodiment, the pharmaceutical combination according to the invention is a pharmaceutical composition, i.e. a fixed-dose combination.

The amount of the PPAR agonist and the p38 inhibitor to be administered will vary depending upon factors such as the particular compound, disease condition and its severity, according to the particular circumstances surrounding the case, including, e.g., the specific PPAR agonist being administered, the route of administration, the condition being treated, the target area being treated, and the subject or host being treated.

In one embodiment, the invention provides a pharmaceutical combination comprising a PPAR agonist and a p38 inhibitor, wherein said PPAR agonist and said p38 inhibitor are present in a therapeutically effective amount.

In a preferred embodiment, the invention provides a pharmaceutical combination comprising a PPAR agonist and a p38 inhibitor, wherein said PPAR agonist and said p38 inhibitor produce an additive therapeutic effect i.e. wherein said PPAR agonist and said p38 inhibitor are present in an amount producing an additive therapeutic effect.

As used herein, the term “additive” means that the effect achieved with the pharmaceutical combinations of this invention is approximately the sum of the effects that result from using the agents, namely the PPAR agonist and the p38 inhibitor, as a monotherapy.

Advantageously, an additive effect provides for greater efficacy at the same doses, and may lead to longer duration of response to the therapy.

In a further preferred embodiment, the invention provides a pharmaceutical combination comprising a PPAR agonist and a p38 inhibitor, wherein said PPAR agonist and said p38 inhibitor produce a synergistic therapeutic effect, i.e. wherein said PPAR agonist and said p38 inhibitor are present in an amount producing a synergistic therapeutic effect.

As used herein, the term “synergistic” means that the effect achieved with the pharmaceutical combinations of this invention is greater than the sum of the effects that result from using the agents, namely the PPAR agonist and the p38 inhibitor, as a monotherapy, i.e. as single agent. Advantageously, such synergy provides greater efficacy at the same doses and may lead to longer duration of response to the therapy. In particular, synergy as referred herein may lead to higher efficacy, to longer duration of response, and to better safety. Better safety may come from the ability to employ lower doses in the combination that may be sufficient to achieve the desired therapeutic effect, which may not be achievable with the same or higher doses of the individual drugs as described in e.g. Drug Synergism: Its Detection and Applications Ronald J. Tallarida. Journal of Pharmacology and Experimental Therapeutics September 2001, 298 (3) 865-872.

In one embodiment, the invention provides a pharmaceutical combination comprising a p38 inhibitor and a PPAR agonist, wherein the amount of said PPAR agonist in the combination is from about 0.1 to about 45 mg, from about 0.1 to about 30 mg or from about 0.1 to about 15 mg, preferably from about 1 to about 30 mg, more preferably from about 1 to about 15 mg, even more preferably from about 5 to about 10 mg.

In a preferred embodiment, the invention provides a pharmaceutical combination comprising a p38 inhibitor and pioglitazone or a pharmaceutically acceptable salt thereof, wherein the amount of pioglitazone or a pharmaceutically acceptable salt thereof in the combination is below the dose typically needed for the treatment of diabetes with pioglitazone or a pharmaceutically acceptable salt thereof.

In one embodiment, the invention provides a pharmaceutical combination comprising a p38 inhibitor and a PPAR agonist, wherein the amount of said p38 inhibitor in the combination is from about 1 to about 500 mg or from about 1 to about 450 mg or from about 1 to about 400 mg or from about 1 to about 350 mg or from about 1 to about 300 mg or from about 1 to about 250 mg or from about 1 to about 200 mg or from about 1 to about 150 mg or from about 1 to about 125 mg or from about 1 to about 100 mg or from about 10 to about 125 mg or from about 10 to about 100 mg or from about 20 to about 100 mg or from about 30 to about 100 mg or from about 40 to about 100 mg or from about 50 to about 100 mg, preferably from about 1 to about 250 mg, more preferably from about 10 to about 200 mg, even more preferably from about 50 to about 150 mg, in particular from about 100 to about 150 mg.

In a preferred embodiment, the pharmaceutical combination of the invention is a pharmaceutical composition (i.e. a fixed-dose combination, as outlined above). In one embodiment, the pharmaceutical combination of the invention is a pharmaceutical composition and includes other medicinal or pharmaceutical agents, e.g., one or more pharmaceutically acceptable diluents, excipients or carriers.

Modes of Administration and Treatment

The terms “treatment”/“treating” as used herein includes: (1) delaying the appearance of clinical symptoms of the state, disorder or condition developing in an animal, particularly a mammal and especially a human, that may be afflicted with or predisposed to the state, disorder or condition but does not yet experience or display clinical or subclinical symptoms of the state, disorder or condition; (2) inhibiting the state, disorder or condition (e.g. arresting, reducing or delaying the progression of the disease, or a relapse thereof in case of maintenance treatment, of at least one clinical or subclinical symptom thereof); and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

Preventive treatments comprise prophylactic treatments. In preventive applications, the pharmaceutical combination of the invention is administered to a subject suspected of having, or being at risk for developing viral diseases or disorders. In therapeutic applications, the pharmaceutical combination of the invention is administered to a subject such as a patient already suffering from viral diseases or disorders, in an amount sufficient to cure or at least partially arrest the severity of the symptoms of the disease. Amounts effective for this use will depend on the severity and course of the disease, previous therapy, the subject's health status and response to the drugs, and the judgment of the treating physician. In the case wherein the subject's condition does not improve, the pharmaceutical combination of the invention may be administered chronically, which is, for an extended period of time, including throughout the duration of the subject's life in order to ameliorate or otherwise control or limit the symptoms of the subject's disease or condition.

In the case wherein the subject's status does improve, the pharmaceutical combination of the invention may be administered continuously; alternatively, the dose of drugs being administered may be temporarily reduced or temporarily suspended for a certain length of time (i.e., a “drug holiday”).

In one embodiment the subject has undergone at least one or more prior therapies comprising administration of a drug compound different from the compounds comprised by the pharmaceutical combination of the present invention e.g. the subject has undergone at least one or more therapies comprising administration of e.g. remdesivir for preventing or treating viral diseases or disorders such as COVID-19 prior to the therapy with the pharmaceutical combination of the present invention.

In one embodiment the subject has been vaccinated with a vaccine against a virus prior to the therapy with the pharmaceutical combination of the present invention. In particular the subject has been vaccinated once, twice or three times with a vaccine against a virus such as e.g. SARS-CoV-2 prior to the therapy with the pharmaceutical combination of the present invention.

In one embodiment the subject has been vaccinated with a vaccine against a virus and has become re-infected with the virus prior to the therapy with the pharmaceutical combination of the present invention. In particular the subject has been vaccinated once, twice or three times with a vaccine against a virus such as e.g. SARS-CoV-2 and has become re-infected with a virus such as e.g. SARS-CoV-2 prior to the therapy with the pharmaceutical combination of the present invention.

The pharmaceutical combination according to the invention is, preferably, suitable for oral, topical, injectable, ocular, local ocular (e.g., subconjunctival, intravitreal, retrobulbar or intracameral), systemic (i.e. enteral or parenteral) administration or suitable for administration by inhalation i.e. the combination is administered locally to the lung. More preferably the pharmaceutical combination according to the invention is suitable for oral, topical, injectable administration and/or administration by inhalation, most preferably suitable for oral administration to a subject and comprises a therapeutically effective amount of the active ingredient(s) and optionally one or more suitable pharmaceutically acceptable diluents, excipients or carriers.

If not indicated otherwise, a pharmaceutical combination according to the invention is prepared in a manner known per se, e.g. by means of conventional mixing, granulating, coating, dissolving or lyophilizing processes. In preparing a combination for an oral dosage form, any of the usual pharmaceutical media may be employed, carriers, diluents, granulating agents, lubricants, binders, disintegrating agents and the like. Because of their ease of administration, tablets and capsules represent the most advantageous oral dosage unit forms, in which case solid pharmaceutical carriers are obviously employed.

In a preferred embodiment, the pharmaceutical combination according to the invention is a combination for oral administration. As indicated above, said pharmaceutical combination for oral administration is preferably a pharmaceutical composition, i.e. a fixed-dose combination.

In one embodiment, the pharmaceutical combination according to the invention is a combination for topical administration. As indicated above, said pharmaceutical combination for topical administration is preferably a pharmaceutical composition, i.e. a fixed-dose combination.

In another preferred embodiment, the pharmaceutical combination according to the invention is a combination for injectable administration. As indicated above, said pharmaceutical combination for injectable administration is preferably a pharmaceutical composition, i.e. a fixed-dose combination.

In one embodiment, the pharmaceutical combination according to the invention is a combination for local ocular administration. As indicated above, said pharmaceutical combination for local ocular administration is preferably a pharmaceutical composition, i.e. a fixed-dose combination.

In another preferred embodiment, the pharmaceutical combination according to the invention is a combination for systemic, i.e. enteral or parenteral administration. As indicated above, said pharmaceutical combination for systemic administration is preferably a pharmaceutical composition, i.e. a fixed-dose combination.

In another preferred embodiment, the pharmaceutical combination according to the invention is a combination for administration by inhalation, for example as a nasal spray, or dry powder or aerosol inhalers. For delivery by inhalation, the active compounds are preferably in the form of microparticles. They may be prepared by a variety of techniques, including spray-drying, freeze-drying and micronisation. Aerosol generation can be carried out using, for example, pressure-driven jet atomizers or ultrasonic atomizers, preferably using propellant-driven metered aerosols or propellant-free administration of micronized active compounds from, for example, inhalation capsules or other “dry powder” delivery systems. Microparticles for delivery by inhalation may be formulated with excipients that aid delivery and release. For example, in a dry powder formulation, microparticles may be formulated with large carrier particles that aid flow from the DPI into the lung. Suitable carrier particles are known, and include lactose particles; they may have a mass median aerodynamic diameter of greater than 90 μm.

The active compounds may be dosed as described depending on the inhaler system used. In addition to the active compounds, the administration forms may additionally contain excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of systems are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described EP-A-0505321). Additionally, the combination of the invention may be delivered in multi-chamber devices thus allowing for separate storage and dosing of the PPAR agonist and the p38 inhibitor according to the invention.

In a more preferred embodiment, the pharmaceutical combination according to the invention is administered orally, topically, by injection or by inhalation, even more preferably orally to the subject.

In a preferred embodiment the viral diseases or disorders is viral infectivity and the pharmaceutical combination according to the invention is a combination for oral administration, i.e. is administered orally. As indicated above, said pharmaceutical combination for oral administration is preferably a pharmaceutical composition, i.e. a fixed-dose combination.

A pharmaceutical combination for oral or systemic i.e. enteral or parenteral administration is, for example, a unit dosage form, such as a tablet, a capsule or a suppository.

In one embodiment, the invention provides a pharmaceutical composition comprising a PPAR agonist, such as pioglitazone and a p38 inhhibitor, such as pamapimod and at least one pharmaceutically acceptable carrier, wherein the composition is a solution or a suspension for ocular administration (i.e. eye drops), or an ophthalmic ointment.

In one embodiment, the invention provides a pharmaceutical composition comprising a PPAR agonist, such as pioglitazone and a p38 inhhibitor, such as pamapimod and at least one pharmaceutically acceptable carrier, wherein the composition is a tablet or a capsule, preferably a tablet.

The unit content of active ingredients in an individual dose need not in itself constitute a therapeutically effective amount, since such an amount can be reached by the administration of a plurality of dosage units. A composition according to the invention may contain, e.g., from about 10% to about 100% of the therapeutically effective amount of the active ingredients.

Where the pharmaceutical combination according to the invention is a combined preparation, said PPAR agonist need not be administered in the same form as said p38 inhibitor. As an example, the PPAR agonist may be administered as a powder by inhalation, while the p38 inhibitor may be administered orally as a tablet or vice versa.

In some embodiments the pharmaceutical combination of the invention is administered to the subject in a dose that comprises a dose of a PPAR agonist which is below the dose needed for the treatment of diabetes using said PPAR agonist. In some embodiments the pharmaceutical combination of the invention is administered to the subject in a dose that comprises a dose of a PPAR agonist which is a factor of 3-9 fold lower than the top dose evaluated and tested for the treatment of diabetes, in particular a factor of 3-9 fold lower than the top dose evaluated and tested for the treatment of diabetes in human. The top dose evaluated and tested for the treatment of diabetes in human, e.g for a PPAR gamma agonist, such as pioglitazone hydrochloride, is usually in the range of about 15-45 mg/day. In some embodiments at the PPAR agonist dose used, the side effects seen in the treatment of diabetes using said PPAR agonist are reduced or not present.

In some embodiments the pharmaceutical combination of the invention is administered to the subject in a dose that comprises a dose of a PPAR agonist which is below the active dose for therapeutically relevant antidiabetic or anti-dyslipidemic effect of the PPAR agonist, in particular a dose that is below the active dose for antidiabetic or anti-dyslipidemic effect of the PPAR agonist in human.

A typical dosing regimen of pioglitazone or a pharmaceutically acceptable salt thereof in the treatment of diabetes includes 15 to 45 mg pioglitazone once-daily.

In some embodiments, the pharmaceutical combination of the invention is administered orally to a human in a dose comprising a dose of a PPAR agonist, usually PPAR gamma agonists, PPAR alpha agonists and/or PPAR alpha/gamma dual agonists, preferably a PPAR gamma agonist and/or a PPAR alpha agonist, more preferably a PPAR gamma agonist and/or a PPAR alpha agonist selected from the group consisting of pioglitazone, rosiglitazone, troglitazone, feonofibrate, bezafibrate and pharmaceutically acceptable salts thereof, even more preferably a PPAR gamma agonist, yet more preferably pioglitazone or a pharmaceutically acceptable salt thereof, most preferably pioglitazone hydrochloride of 0.1-45 mg/day, preferably 0.1-10 mg/day, more preferably about 5 mg/day; and comprising a dose of a p38 inhibitor, e.g. a compound of formula I or II, in particular a compound of formula I, preferably a p38 inhibitor selected from the group consisting of pamapimod, acumapimod, losmapimod, dilmapimod, semapimod, AZD7624, ARRY-371797, LY2228820, R⁹¹¹¹, PH-797804, BIRB 796, VX-702, VX-745 SB 239063, SB202190, SCIO 469, and BMS 582949 or a pharmaceutically acceptable salt thereof, more preferably pamapimod or a pharmaceutically acceptable salt thereof of 1-500 mg/day, preferably 10-250 mg/day, more preferably 25-150 mg/day, most preferably about 75 mg/day.

In some embodiments, the pharmaceutical combination of the invention is administered orally to a human in a dose comprising a dose of a PPAR agonist, usually PPAR gamma agonists, PPAR alpha agonists and/or PPAR alpha/gamma dual agonists, preferably a PPAR gamma agonist and/or a PPAR alpha agonist, more preferably a PPAR gamma agonist and/or a PPAR alpha agonist selected from the group consisting of pioglitazone, rosiglitazone, troglitazone, feonofibrate, bezafibrate and pharmaceutically acceptable salts thereof, even more preferably a PPAR gamma agonist, yet more preferably pioglitazone or a pharmaceutically acceptable salt thereof, most preferably pioglitazone hydrochloride of 0.1-45 mg/day, preferably 0.1-20 mg/day, more preferably about 10 mg/day; and comprising a dose of a p38 inhibitor, e.g. a compound of formula I or II, in particular a compound of formula I, preferably a p38 inhibitor selected from the group consisting of pamapimod, acumapimod, losmapimod, dilmapimod, semapimod, AZD7624, ARRY-371797, LY2228820, R⁹¹¹¹, PH-797804, BIRB 796, VX-702, VX-745 SB 239063, SB202190, SCIO 469, and BMS 582949 or a pharmaceutically acceptable salt thereof, more preferably pamapimod or a pharmaceutically acceptable salt thereof of 1-500 mg/day, preferably 10-250 mg/day, more preferably 25-150 mg/day, most preferably about 150 mg/day.

In some embodiments, the pharmaceutical combination of the invention is administered orally to a human in a dose comprising a dose of a PPAR agonist, usually PPAR gamma agonists, PPAR alpha agonists and/or PPAR alpha/gamma dual agonists, preferably a PPAR gamma agonist and/or a PPAR alpha agonist, more preferably a PPAR gamma agonist and/or a PPAR alpha agonist selected from the group consisting of pioglitazone, rosiglitazone, troglitazone, feonofibrate, bezafibrate and pharmaceutically acceptable salts thereof, even more preferably a PPAR gamma agonist, yet more preferably pioglitazone or a pharmaceutically acceptable salt thereof, most preferably pioglitazone hydrochloride of 0.1-45 mg, preferably 0.1-10 mg, more preferably about 5 mg once daily, twice daily, or thrice daily, preferably twice daily; and comprising a dose of a p38 inhibitor, e.g. a compound of formula I or II, in particular a compound of formula I, preferably a p38 inhibitor selected from the group consisting of pamapimod, acumapimod, losmapimod, dilmapimod, semapimod, AZD7624, ARRY-371797, LY2228820, R⁹¹¹¹, PH-797804, BIRB 796, VX-702, VX-745 SB 239063, SB202190, SCIO 469, and BMS 582949 or a pharmaceutically acceptable salt thereof, more preferably pamapimod or a pharmaceutically acceptable salt thereof of 1-500 mg, preferably 10-250 mg, more preferably 25-150 mg, most preferably about 75 mg/day once daily, twice daily, or thrice daily, preferably twice daily.

Dosing Regimen

An exemplary treatment regime entails administration once daily, twice daily, or thrice daily every second day, preferably once daily and/or twice daily. The combination of the invention is usually administered on multiple occasions. Intervals between single dosages can be, for example, less than a day, daily, or every second day. The combination of the invention may be given as a continous uninterrupted treatment. The combination of the invention may also be given in a regime in which the subject receives cycles of treatment interrupted by a drug holiday or period of non-treatment. Thus, the combination of the invention may be administered according to the selected intervals above for a continuous period of one week or a part thereof, for two weeks, for three weeks, for four weeks, for five weeks or for six weeks and then stopped for a period of one week, or a part thereof, for two weeks, for three weeks, for four weeks, for five weeks, or for six weeks. The combination of the treament interval and the non-treatment interval is called a cycle. The cycle may be repeated one or more times. Two or more different cycles may be used in combination for repeating the treatment one or more times. Intervals can also be irregular and guided either by worseining or improvement in the condition of the patient indicated by appearance or remission of symptoms or objective evidence of disease appearance or remission. In such case, therapy may be started and suspended as needed, and only restarted when symptoms or objective measures indicate the return of disease. In a preferred embodiment, the pharmaceutical combination according to the invention is administered once daily or twice daily.

Kits/Articles of Manufacture

In one aspect, the present invention also provides a kit for use in a method of preventing or treating viral diseases or disorders in a subject, comprising a pharmaceutical combination disclosed herein, and instructions for using the kit. Preferred PPAR agonists and preferred p38 kinase inhibitors comprised by said pharmaceutical combination are as described above.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic. The articles of manufacture provided herein generally will comprise one or more pharmaceutical combination disclosed herein and packaging materials. Examples of pharmaceutical packaging materials include, but are not limited to, blister packs, bottles, tubes, inhalers, pumps, bags, vials, containers, syringes, and any packaging material suitable for a selected composition and intended mode of administration and treatment.

Preventing or Treating Viral Diseases or Disorders

In one aspect, the present invention provides a pharmaceutical combination described herein, i.e. a pharmaceutical combination comprising:
(a) a PPAR agonist;
(b) a p38 kinase inhibitor; and optionally
(c) one or more pharmaceutically acceptable diluents, excipients or carriers for use in a method of preventing or treating viral diseases or disorders in a subject.

Also provided is the use of a pharmaceutical combination described herein for the manufacture of a medicament for preventing or treating viral diseases or disorders in a subject.

Also provided is the use of a pharmaceutical combination described herein for preventing or treating viral diseases or disorders in a subject.

Also provided is a method of preventing or treating viral diseases or disorders in a subject, comprising administering to said subject a therapeutically effective amount of a pharmaceutical combination as described herein.

In an embodiment, the present invention provides a pharmaceutical combination described herein for use in a method of preventing or treating viral infectivity or viral shedding.

In a preferred embodiment, the present invention provides a pharmaceutical combination described herein for use in a method of preventing or treating viral diseases or disorders in a subject, wherein said viral diseases or disorders are caused by viruses affecting the lung.

In a more preferred embodiment, the present invention provides a pharmaceutical combination described herein for use in a method of preventing or treating viral diseases or disorders in a subject, wherein said viral diseases or disorders are caused by a virus selected from the group consisting of SARS-CoV-2 (Coronavirus associated with COVID-19); SARS-CoV(Coronavirus associated with SARS); HCoV (human coronavirus); HA and NA influenza viruses; ADV (adenovirus); HBoV (human bocavirus); HMPV (human metapneumovirus); HPIV (human parainfluenza virus); HRSV (human respiratory syncytial virus); and HRV (human rhinovirus).

In an even more preferred embodiment, the present invention provides a pharmaceutical combination described herein for use in a method of preventing or treating viral diseases or disorders in a subject, wherein said viral diseases or disorders are caused by a coronavirus, preferably by SARS-CoV-2.

In a particular preferred embodiment, the present invention provides a pharmaceutical combination described herein for use in a method of preventing or treating viral diseases or disorders in a subject, wherein said viral disease or disorder is COVID-19.

In an more particular embodiment, the present invention provides a pharmaceutical combination described herein for use in a method of preventing or treating viral diseases or disorders in a subject, wherein said viral diseases or disorders are caused by a coronavirus, preferably by SARS-CoV-2 and wherein the patient population is selected from the group consisting of SARS-CoV-2 positive hospitalized patients, SARS-CoV-2 asymptomatic patients, SARS-CoV-2 positive patients with mild symptoms not requiring hospitalization and patients with resolved SARS-CoV-2 infection.

In a further more particular preferred embodiment, the present invention provides a pharmaceutical combination described herein for use in a method of preventing or treating viral diseases or disorders in a subject, wherein said viral disease or disorder is COVID-19 and wherein the patient population is selected from the group consisting of SARS-CoV-2 positive hospitalized patients, SARS-CoV-2 asymptomatic patients, SARS-CoV-2 positive patients with mild symptoms not requiring hospitalization and patients with resolved SARS-CoV-2 infection.

In a more preferred embodiment, the present invention provides a pharmaceutical combination described herein for use in a method of preventing or viral infectivity or shedding.

In one embodiment, the present invention provides a pharmaceutical combination comprising:
(a) a PPAR agonist;
(b) a p38 kinase inhibitor; and optionally
(c) one or more pharmaceutically acceptable diluents, excipients or carriers for use in a method of preventing or treating viral diseases or disorders in a subject, wherein said p38 kinase inhibitor is preferably inhibiting p38-alpha, p38-beta, p38-gamma or p38-delta or combinations thereof, more preferably inhibiting p38-alpha and/or p38-beta.

In one embodiment, the present invention provides a pharmaceutical combination comprising:
(a) a PPAR agonist;
(b) a p38 kinase inhibitor; and optionally
(c) one or more pharmaceutically acceptable diluents, excipients or carriers for use in a method of preventing or viral diseases or disorders in a subject, wherein said p38 kinase inhibitor is selected from the group consisting of pamapimod, acumapimod, losmapimod, dilmapimod, semapimod, AZD7624, ARRY-371797, LY2228820, R⁹¹¹¹, PH-797804, BIRB 796, VX-702, VX-745 SB 239063, SB202190, SCIO 469, and BMS 582949 or a pharmaceutically acceptable salt thereof.

In one embodiment, the present invention provides a pharmaceutical combination comprising:
(a) a PPAR agonist;
(b) a p38 kinase inhibitor; and optionally
(c) one or more pharmaceutically acceptable diluents, excipients or carriers for use in a method of preventing or viral diseases or disorders in a subject, wherein said p38 kinase inhibitor is selected from the group consisting of a compound of formula I or a pharmaceutically acceptable salt thereof, losmapimod, semapimod, AZD7624, ARRY-371797, LY2228820, R⁹¹¹¹, PH-797804, BIRB 796, VX-702, VX-745, SB 239063, SB202190, SCIO 469, and BMS 582949, and a pharmaceutically acceptable salt thereof.

In a further embodiment, the present invention provides a pharmaceutical combination according to the invention, comprising
(a) a PPAR agonist;
(b) a compound of formula I or a pharmaceutically acceptable salt thereof as defined herein; and optionally
(c) one or more pharmaceutically acceptable diluents, excipients or carriers for use in a method of preventing or treating viral diseases or disorders in a subject.

In a further embodiment, the present invention provides a pharmaceutical combination comprising
(a) a PPAR agonist;
(b) a compound of formula II or a pharmaceutically acceptable salt thereof as defined herein; and optionally
(c) one or more pharmaceutically acceptable diluents, excipients or carriers for use in a method of preventing or treating viral diseases or disorders in a subject.

In a preferred embodiment, the present invention provides a pharmaceutical combination comprising:
(a) a PPAR agonist;
(b) pamapimod or a pharmaceutically acceptable salt thereof; and optionally
(c) one or more pharmaceutically acceptable diluents, excipients or carriers for use in a method of preventing or treating viral diseases or disorders in a subject.

In a further embodiment, the present invention provides a pharmaceutical combination comprising
(a) a PPAR gamma agonist;
(b) a compound of formula I or a pharmaceutically acceptable salt thereof as defined herein; and optionally
(c) one or more pharmaceutically acceptable diluents, excipients or carriers for use in a method of preventing or treating viral diseases or disorders in a subject.

In a further embodiment, the present invention provides a pharmaceutical combination comprising
(a) a PPAR gamma agonist;
(b) a compound of formula II or a pharmaceutically acceptable salt thereof as defined herein; and optionally
(c) one or more pharmaceutically acceptable diluents, excipients or carriers for use in a method of preventing or treating viral diseases or disorders in a subject.

In a preferred embodiment, the present invention provides a pharmaceutical combination comprising
(a) a PPAR gamma agonist;
(b) a compound of formula I or a pharmaceutically acceptable salt thereof as defined herein; and optionally
(c) one or more pharmaceutically acceptable diluents, excipients or carriers for use in a method of preventing or treating viral diseases or disorders in a subject;

wherein said PPAR gamma agonist is selected from the group consisting of pioglitazone, rosiglitazone, troglitazone and INT131 or a pharmaceutically acceptable salt thereof; and

wherein X¹ and X² in said compound of formula I are each 0; and

wherein Z in said compound of formula I is N; and

wherein W in said compound of formula I is NH; and

wherein Ar¹ in said compound of formula I is aryl; and

wherein R¹ in said compound of formula I is heteroalkyl; and

wherein R³ in said compound of formula I is alkyl.

In a further preferred embodiment, the present invention provides a pharmaceutical combination comprising
(a) a PPAR gamma agonist;
(b) pamapimod, R⁹¹¹¹, semapimod, or a pharmaceutically acceptable salt thereof, preferably pamapimod or a pharmaceutically acceptable salt thereof; and optionally
(c) one or more pharmaceutically acceptable diluents, excipients or carriers for use in a method of preventing or treating viral diseases or disorders in a subject; wherein said PPAR gamma agonist is selected from the group consisting of pioglitazone, troglitazone, bezafibrate and pharmaceutically acceptable salts thereof.

In a further preferred embodiment, the present invention provides a pharmaceutical combination comprising
(a) a PPAR gamma agonist;
(b) pamapimod, losmapimod, ARRY-371797, or a pharmaceutically acceptable salt thereof, preferably pamapimod or a pharmaceutically acceptable salt thereof, and optionally
(c) one or more pharmaceutically acceptable diluents, excipients or carriers for use in a method of preventing or treating viral diseases or disorders in a subject; wherein said PPAR gamma agonist is selected from the group consisting of pioglitazone, troglitazone, bezafibrate and pharmaceutically acceptable salts thereof.

In one embodiment, the present invention provides a pharmaceutical combination comprising:
(a) a PPAR agonist;
(b) a p38 kinase inhibitor; and optionally
(c) one or more pharmaceutically acceptable diluents, excipients or carriers for use in a method of preventing or treating viral diseases or disorders in a subject, wherein said PPAR agonist is activating PPAR alpha, PPAR gamma or PPAR delta or combinations thereof.

In one embodiment, the present invention provides a pharmaceutical combination comprising:
(a) a PPAR agonist;
(b) a p38 kinase inhibitor; and optionally
(c) one or more pharmaceutically acceptable diluents, excipients or carriers for use in a method of preventing or treating viral diseases or disorders in a subject, wherein said PPAR agonist is selected from the group consisting of pioglitazone, troglitazone, rosiglitazone, bezafibrate, fenofibrate, clofibrate, gemfibrozil, aleglitazar, muraglitazar, tesaglitazar, ragaglitazar, saroglitazar, GFT505, naveglitazar, GW501516 and INT131 or a pharmaceutically acceptable salt thereof.

In one embodiment, the present invention provides a pharmaceutical combination comprising:
(a) a PPAR agonist;
(b) a p38 kinase inhibitor; and optionally
(c) one or more pharmaceutically acceptable diluents, excipients or carriers for use in a method of preventing or treating viral diseases or disorders in a subject, wherein said PPAR agonist is selected from the group consisting of pioglitazone, troglitazone, bezafibrate and pharmaceutically acceptable salts thereof.

In a preferred embodiment, the present invention provides a pharmaceutical combination comprising:
(a) a PPAR agonist;
(b) a p38 kinase inhibitor; and optionally
(c) one or more pharmaceutically acceptable diluents, excipients or carriers for use in a method of preventing or treating viral diseases or disorders in a subject, wherein said PPAR agonist is pioglitazone or a pharmaceutically acceptable salt thereof.

In a further preferred embodiment, the present invention provides a pharmaceutical combination comprising:
(a) pioglitazone or a pharmaceutically acceptable salt thereof,
(b) a p38 kinase inhibitor; and optionally
(c) one or more pharmaceutically acceptable diluents, excipients or carriers for use in a method of preventing or treating viral diseases or disorders in a subject.

In a further preferred embodiment, the present invention provides a pharmaceutical combination comprising:
(a) pioglitazone hydrochloride;
(b) a p38 kinase inhibitor; and optionally
(c) one or more pharmaceutically acceptable diluents, excipients or carriers for use in a method of preventing or treating viral diseases or disorders in a subject.

In a particularly preferred embodiment, the present invention provides a pharmaceutical combination comprising:
(a) pioglitazone or a pharmaceutically acceptable salt thereof, preferably pioglitazone hydrochloride;
(b) pamapimod, losmapimod, ARRY-371797, or a pharmaceutically acceptable salt thereof; and optionally
(c) one or more pharmaceutically acceptable diluents, excipients or carriers for use in a method of preventing or treating viral diseases or disorders in a subject.

In a particularly preferred embodiment, the present invention provides a pharmaceutical combination comprising:
(a) pioglitazone or a pharmaceutically acceptable salt thereof, preferably pioglitazone hydrochloride;
(b) pamapimod or a pharmaceutically acceptable salt thereof; and optionally
(c) one or more pharmaceutically acceptable diluents, excipients or carriers for use in a method of preventing or treating viral diseases or disorders in a subject.

In a further particularly preferred embodiment, the present invention provides a pharmaceutical combination comprising:
(a) pioglitazone or a pharmaceutically acceptable salt thereof, preferably pioglitazone hydrochloride;
(b) pamapimod or a pharmaceutically acceptable salt thereof; and optionally
(c) one or more pharmaceutically acceptable diluents, excipients or carriers for use in a method of preventing or treating viral diseases or disorders in a subject, wherein said viral diseases or disorders is COVID-19.

### EXAMPLES

The present Examples are intended to illustrate the present invention without restricting it.

### Example 1: Combination Treatment with Pamapimod and Pioglitazone Vs. The Single Agents on Gene Expression in Mouse Lung

1. Summary

Viral infection consists of receptor-mediated uptake of viral particles, uncoating, activation of cell signaling events leading to viral replication, virus assembly and exocytosis. Several genes participate in the viral lifecycle and also the resulting damages and death of host cells. Whole genome expression analysis on lung tissue from mice orally treated with the agents was performed. Surprisingly, the results revealed that pioglitazone and pamapimod have a synergistic effect to significantly regulate the expression of more genes involved in the viral lifecycle and host cell responses than either drug alone. These data suggest that the combination of the two drugs may provide better therapeutic efficacy in the treatment of viral infection and its consequences than either drug alone.

2. Materials and Methods

Global gene expression changes were determined by Illumina Next Generation RNA sequencing in lung samples from mice treated orally for 3 weeks with pioglitazone or pamapimod or the combination of pioglitazone+pamapimod. RNA was extracted from the post caval lobe that had been snap frozen at the termination of the study. For this analysis, 8 samples from each of treatment groups were analysed. Briefly, total RNA was extracted for next generation sequencing using standard methods, then Illumina TruSeq RNA libraries including poly(A) enrichment were prepared. Sequencing was performed on an Illumina NextSeq 500, v2, high output, 1×75 bp reads with 30 Mio packages. Demultiplexing and trimming of Illumina adaptor residuals was performed on the raw data. Mapping of data reads was made using the reference mouse genome mm10.

For bioinformatics analysis, the average expression level and standard deviations were determined for each gene for the 8 replicates per treatment group. Pairwise comparisons between groups were then performed to identify differentially expressed genes. Absolute p-values and adjusted p-values, to correct for multiplicity of testing, were calculated for each pairwise analysis. Data output consisted of the top 1000 differentially expressed genes. A gene was considered significantly up or downregulated if the difference in a between-group comparison yielded an adjusted p value<0.10.

3. Results

Table 1 below shows the significantly regulated genes (adjusted p value<0.10) for the three treatment groups in comparison to a control untreated group. Pioglitazone or pamapimod single regulated only a few genes implicated in viral replication and host responses. Surprisingly, combined treatment with pioglitazone/pamapimod favorably altered expression of many additional genes implicated in the pathogenesis of viral infection and host cell response, compared to either no effect or minor effects of the agents alone. These data strongly support synergy of the combination, indicating that the combination has potentially greater benefit in treating viral infections and improving outcomes for patients.

TABLE 1
Synergistic effect of the pamapimod/pioglitazone
combination on gene expression in mouse liver.

| | Adjusted p value (significance)* | | | |
|---|---|---|---|---|
| Gene | Pioglit- | | Pio/ | |
| ID | azone | Pamapimod | PamCombination | Direction |
| Il6 | n.s. | 2.48e−01 | 1.61e−04 | downregulated |
| Ankrd37 | n.s. | 3.57e−01 | 1.35e−02 | downregulated |
| Tnfrsf11b | n.s. | n.s. | 2.84e−03 | downregulated |
| Il12b | n.s. | 2.27e−02 | 1.42e−02 | downregulated |
| Ccr5 | n.s. | n.s. | 1.61e−02 | downregulated |
| Dnajb1 | n.s. | 2.45e−01 | 1.34e−02 | downregulated |
| Dnaja1 | n.s. | 2.52e−01 | 4.10e−02 | downregulated |
| Adamts9 | 7.58e−01 | n.s. | 1.15e−01 | downregulated |
| Pprc1 | n.s. | n.s. | 1.06e−01 | downregulated |
| Ccdc117 | n.s. | n.s. | 6.88e−02 | downregulated |
| Rasl11a | n.s. | n.s. | 1.07e−01 | downregulated |
| Foxd4 | n.s. | n.s. | 4.42e−02 | upregulated |
| Apol11b | n.s. | n.s. | 3.67e−03 | upregulated |
| Mmp9 | n.s. | n.s. | 5.41e−02 | upregulated |
| Fkbp5 | n.s. | 2.46e−01 | 3.68e−03 | upregulated |
| Fabp1 | 7.58e−01 | n.s. | 9.72e−02 | upregulated |
| Nr1d1 | 9.06e−01 | n.s. | 2.65e−03 | upregulated |
| Cidec | 8.10e−01 | n.s. | 9.21e−02 | upregulated |
| Nr1d2 | 3.68e−01 | 2.14e−02 | 1.89e−06 | upregulated |
| Per3 | n.s. | n.s. | 4.61e−03 | upregulated |
| Sh3d21 | n.s. | 7.65e−04 | 4.23e−04 | upregulated |
| Ppargc1a | n.s. | n.s. | 1.05e−01 | upregulated |
| Insig1 | n.s. | 1.83e−04 | 1.43e−02 | upregulated |
| Pik3ip1 | n.s. | 3.09e−02 | 1.67e−02 | upregulated |

*n.s. = adjusted p value > 0.10

### Example 2: Pamapimod Inhibits Replication of SARs-CoV-2 in the Vero-4 Kidney Epithelial Cell Line in Culture

1. Summary

Viral infection consists of receptor-mediated uptake of viral particles, uncoating, activation of cell signaling events leading to viral replication, virus assembly and shedding. Several genes participate in the viral lifecycle. Cultured Vero-4 kidney epithelial cells were infected with SARS-CoV-2 viruses. Surprisingly, the results revealed that pamapimod inhibits shedding of newly synthesized and packaged viral particles into the culture supernatant. These data suggest that pamapimod is effective as an antiviral agent. In light of the data presented in example 1, the therapeutic efficacy in the treatment of viral infection for the pamapimod/pioglitazone combination is expected to be synergistic.

2. Materials and Methods

Vero B4 cells were infected with SARS-CoV-2PR1 for 1 hr, input virus removed, cells washed, and seeded in 6 well plates. Compounds at the indicated concentrations were added and cells treated for 72 hrs. Supernatant samples, containing released viruses, were evaluated for viral RNA by qRT-PCR. Released virus particles were purified by sucrose centrifugation, proteins separated by SDS-PAGE, and SARS-CoV-2 nucleocapsid protein detected by Western Blot analysis. Cell viability was determined by WST assay. Remdesivir was used as an experimental control.

3. Results

FIG. 1 shows the effect of pamapimod, at decreasing concentrations, to inhibit shedding of new SARS-CoV-2 viruses into culture supernatants, as determined by qRT-PCR. Pamapimod showed antiviral activity with 80% inhibition at the lowest concentration tested (100 nM).

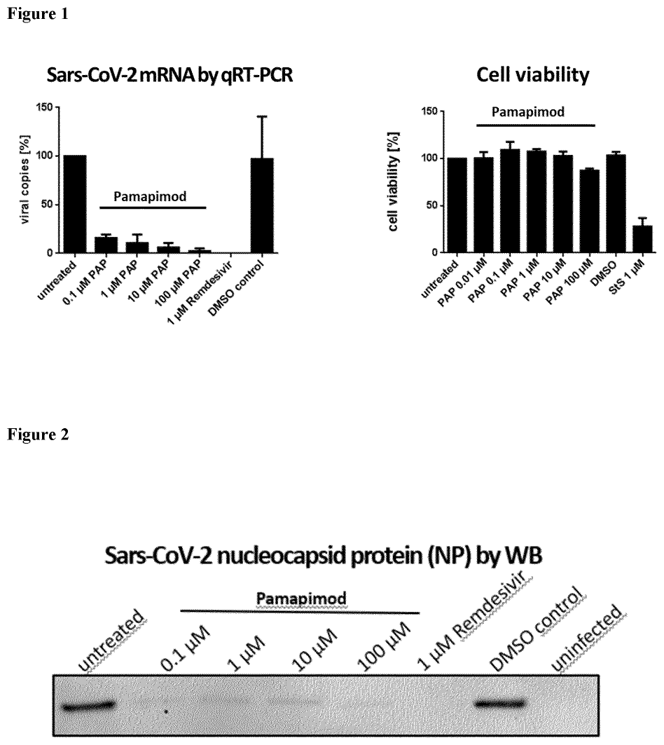

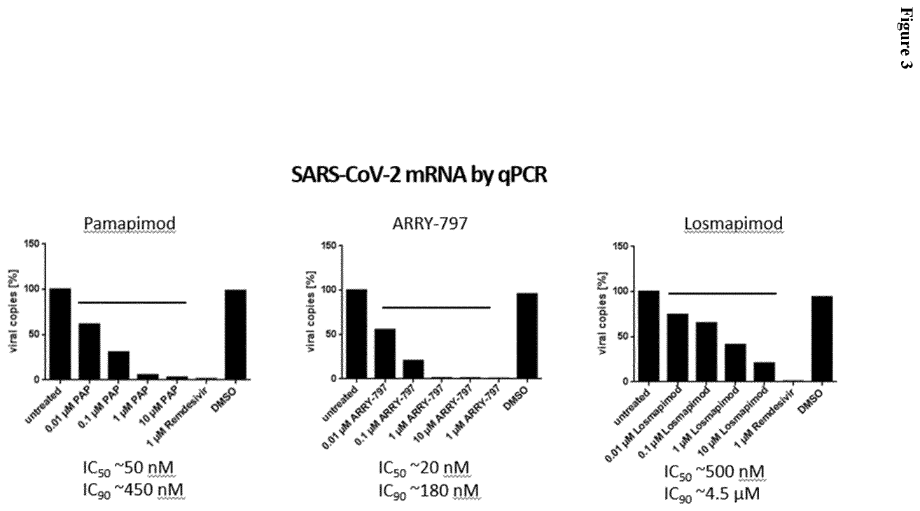

These data were confirmed by measurement of new virus particles in the supernatant, as determined by Western Blot analysis detecting the SARS-CoV-2 nucleocapsid protein (FIG. 2). Pamapimod showed no cellular toxicity as determined by WST assay (FIG. 1). Remdesivir was used as positive control.

### Example 3: Other p38MAPK Inhibitors Inhibit SARS-CoV-2 Replication

1. Summary

Cultured Vero-B4 kidney epithelial cells were infected with SARS-CoV-2 viruses and treated with compounds. Surprisingly, the results revealed that all p38MAPK inhibitors tested inhibited shedding of newly synthesized and packaged viral particles into the culture supernatant. These data suggest that p38MAPK inhibitors as a drug class are effective antivirals against SARS-CoV-2.

2. Materials and Methods

Vero B4 cells were infected with SARS-CoV-2PR1 for 1 hr, input virus removed, cells washed, and seeded in 6 well plates. Compounds at the indicated concentrations were added and cells treated for 72 hrs. Supernatant samples, containing released viruses, were evaluated for viral RNA by qRT-PCR. Remdesivir was used as an experimental control.

3. Results

FIG. 3 shows the effect of pamapimod, ARRY-797, and losmapimod at decreasing concentrations to inhibit shedding of new SARS-CoV-2 viruses into culture supernatants, as determined by qRT-PCR. All three p38MAPK inhibitors showed high antiviral activity with >90% inhibition of viral shedding at the highest concentrations. These data suggest that p38MAPK inhibitors in general, due to sharing a common mechanism, are highly effective as antiviral agents.

### Example 4: PPARγ Agonist Pioglitazone Inhibits SARS-CoV-2 Replication

1. Summary

Cultured Vero-B4 kidney epithelial cells were infected with SARS-CoV-2 viruses and treated with compounds. Surprisingly, the results revealed that the PPARγ agonist pioglitazone inhibited shedding of newly synthesized and packaged viral particles into the culture supernatant. These data suggest that PPARγ agonists such as pioglitazone are effective antivirals against SARS-CoV-2

2. Materials and Methods

Vero B4 cells were infected with SARS-CoV-2PR1 for 1 hr, input virus removed, cells washed, and seeded in 6 well plates. Compounds at the indicated concentrations were added and cells treated for 72 hrs. Remdesivir was used as an experimental control.

3. Results

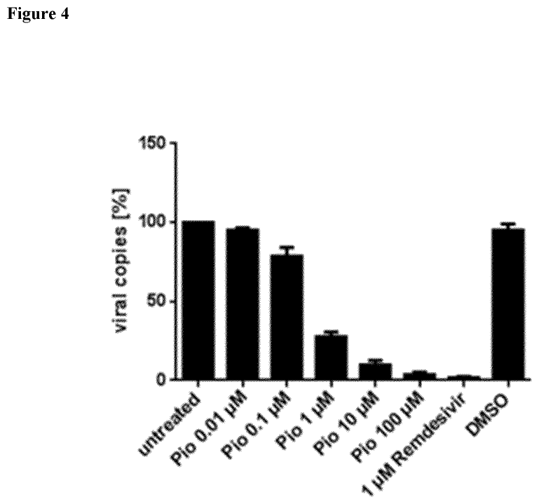

FIG. 4 shows the effect of pioglitazone, a PPARγ agonist at decreasing concentrations, to inhibit shedding of new SARS-CoV-2 viruses into culture supernatants, as determined by qRT-PCR. Pioglitazone showed high antiviral activity with >90% inhibition of viral shedding at the highest concentration. Surprisingly, the results revealed that pioglitazone significantly inhibits shedding of newly synthesized and packaged viral particles into the culture supernatant. These data suggest that PPARγ agonists are highly effective antiviral agents against SARS-CoV-2.

### Example 5: Combination Treatment with Pamapimod and Pioglitazone Vs. The Single Compounds Synergistically Inhibits SARS-CoV-2 Replication

1. Summary

Cultured Vero-B4 kidney epithelial cells were infected with SARS-CoV-2 viruses and treated with compounds. Surprisingly, the results revealed that combining pamapimod, a p38MAPK inhibitor with pioglitazone, a PPARγ agonist, synergistically inhibited shedding of newly synthesized and packaged viral particles into the culture supernatant. The effects of combination treatment were much greater than the sum of the effects of the single drugs alone. These data suggest that combining p38MAPK inhibitors with PPARγ agonists are effective antivirals against SARS-CoV-2 due to drug synergy.

2. Materials and Methods

Vero B4 cells were infected with SARS-CoV-2PR1 for 1 hr, input virus removed, cells washed, and seeded in 6 well plates. Compounds at the indicated concentrations were added and cells treated for 72 hrs. Supernatant samples, containing released viruses, were evaluated for viral RNA by qRT-PCR. Remdesivir was used as an experimental control.

3. Results

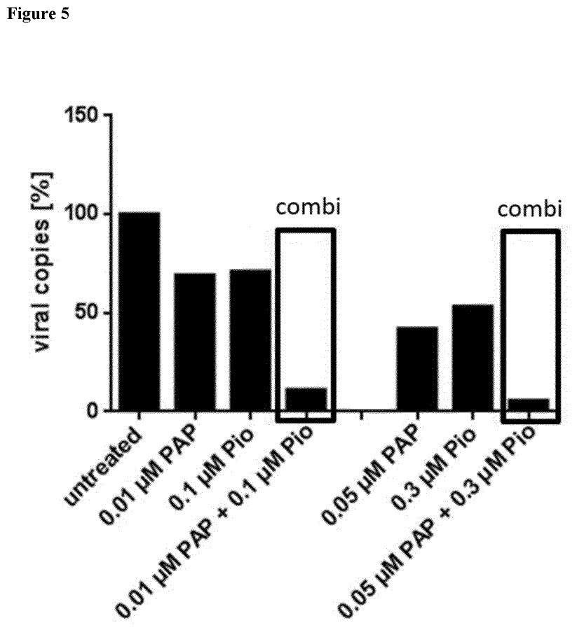

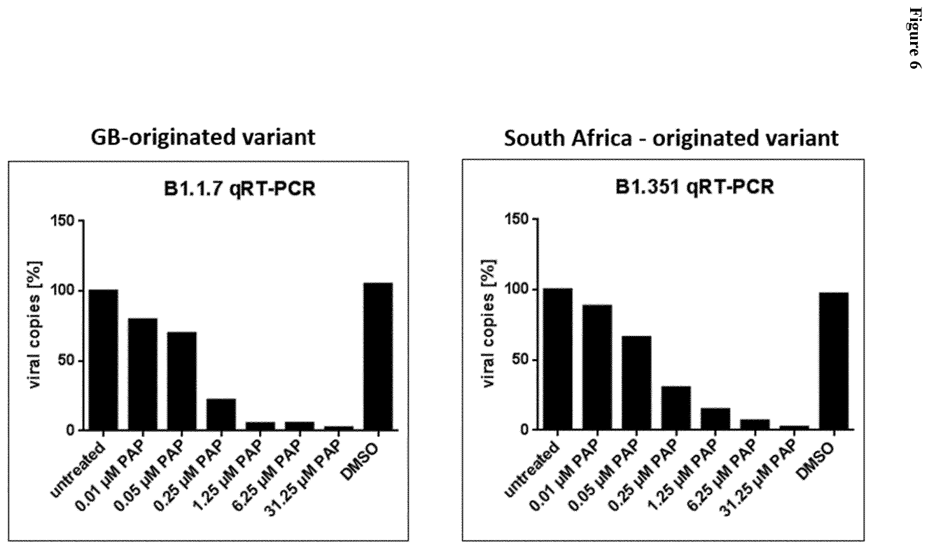

FIG. 5 shows the effects of combining pamapimod, a p38MAPK inhibitor, with pioglitazone, a PPARγ agonist on antiviral activity against SARS-CoV-2. The concentrations were chosen to match the IC₂₀ and IC₅₀ concentrations determined for the drugs alone. In light of the already surprising significant effects of the single agents, the combination of the two agents did not show simply an additive effect, but very surprisingly demonstrated an extremely high level of synergy to inhibit shedding of new SARS-CoV-2 viruses, as determined by qRT-PCR. These data demonstrate that combinations of p38MAPK inhibitors, such as pamapimod, and PPARγ agonists, such as pioglitazone demonstrate synergistic antiviral activity.

### Example 6: Pamapimod Inhibits Replication of SARS-CoV-2 Variants

1. Summary

Cultured Vero-B4 kidney epithelial cells were infected with SARS-CoV-2 viruses and treated with compounds. Surprisingly, the results revealed that pamapimod significantly inhibited the activity of SARS-CoV-2 virus variants as reflected by inhibition of shedding of newly synthesized and packaged viral particles into the culture supernatant. These data suggest that p38MAPK inhibitors, such as pamapimod, are highly effective as antiviral agents, not only against the wild-type virus, but against emerging mutant variants.

2. Materials and Methods

Vero B4 cells were infected with SARS-CoV-2B.1.1.7 variant or the SARS-CoV-2 B1.351 variant for 1 hr, input virus removed, cells washed, and seeded in 6 well plates. Compounds at the indicated concentrations were added and cells treated for 72 hrs. Supernatant samples, containing released viruses, were evaluated for viral RNA by qRT-PCR.

3. Results

FIG. 6 shows the effect of pamapimod at decreasing concentrations, to inhibit replication of SARS-CoV-2 virus variants, as determined by qRT-PCR. Pamapimod showed high activity against variants of the virus with >90% inhibition of viral shedding at the highest concentrations. These data indicate that p38MAPK inhibitors are effective antivirals against emerging variants of SARS-CoV-2.

1. A pharmaceutical combination comprising:
(a) a PPAR agonist;
(b) a p38 kinase inhibitor; and optionally
(c) one or more pharmaceutically acceptable diluents, excipients or carriers for use in a method of preventing or treating viral diseases or disorders in a subject.

2. The pharmaceutical combination for use according to claim 1, wherein said p38 kinase inhibitor is inhibiting p38-alpha and/or p38-beta.

3. The pharmaceutical combination for use according to claim 1, wherein said p38 inhibitor is a compound of formula I or II 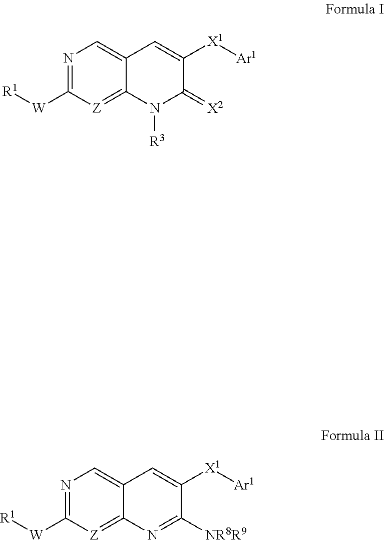
or a pharmaceutically acceptable salt thereof, wherein
Z is N or CH;
W is NR²
X¹ is O, NR⁴ (where R⁴ is hydrogen or alkyl), S, or CR⁵R⁶(where R⁵ and R⁶ are independently hydrogen or alkyl) or C═O;
X² is O or NR⁷;
Ar¹ is aryl or heteroaryl;
R² is hydrogen, alkyl, acyl, alkoxycarbonyl, aryloxycarbonyl, heteroalkylcarbonyl, heteroalkyloxycarbonyl or —R²—R² where R²¹ is alkylene or —C(═O)— and R²² is alkyl or alkoxy;
R¹ is hydrogen, alkyl, haloalkyl, aryl, aralkyl, heteroaryl, heteroaralkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl, R¹²—SO₂-heterocycloamino (where R¹² is haloalkyl, aryl, aralkyl, heteroaryl or heteroaralkyl),—Y¹—C(O)—Y²-R¹¹ (where Y¹ and Y² are independently either absent or an alkylene group and R¹¹ is hydrogen, alkyl, haloalkyl, hydroxy, alkoxy, amino, monoalkylamino or dialkylamino), (heterocyclyl)(cycloalkyl)alkyl or (heterocyclyl)(heteroaryl)alkyl;
R³ is hydrogen, alkyl, cycloalkyl, cycloalkylalkyl, aryl, aralkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino), amino, monoalkylamino, dialkylamino or NR³-Y³-R³³ (where Y³ is —C(O), —C(O)O—, —C(O)NR³⁴, S(O)₂ or S(O)₂NR⁵; R², R³⁴ and R³⁵ are independently hydrogen or alkyl; and R³³ is hydrogen, alkyl, cycloalkyl, cycloalkylalkyl, heteroalkyl or optionally substituted phenyl) or acyl;
R⁷ is hydrogen or alkyl; and
R⁸ and R⁹ are independently hydrogen, alkyl, aryl, aralkyl, cycloalkyl, cycloalkylalkyl, heteroalkyl, alkylsulfonyl, arylsulfonyl, —C(O)—R¹ (where R⁸¹ is alkyl, aryl, aralkyl, cycloalkyl, cycloalkylalkyl, heteroalkyl, alkoxy, aryloxy, amino, mono- or dialkylamino, arylamino or aryl(alkyl)amino) or R⁸ and R⁹ together form=CR⁸2R⁸3 (where R⁸² and R⁸³ are independently hydrogen, alkyl, cycloalkyl, cycloalkylalkyl or optionally substituted phenyl) and optionally one or more pharmaceutically acceptable diluents, excipients or carriers.

4. The pharmaceutical combination for use according to claim 3, wherein said p38 inhibitor is a compound of formula I or a pharmaceutically acceptable salt thereof. 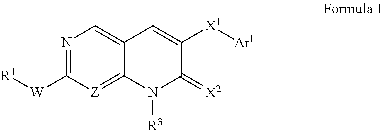
wherein Ar¹, W, X¹, X², Z, R¹ and R³ are as defined in claim 3.

5. The pharmaceutical combination for use according to claim 1, wherein said p38 kinase inhibitor is selected from the group consisting of pamapimod, acumapimod, losmapimod, dilmapimod, semapimod, AZD7624, ARRY-371797, LY2228820, R⁹¹¹¹, PH-797804, BIRB 796, VX-702, VX-745, SB 239063, SB202190, SCIO 469, BMS 582949 and pharmaceutically acceptable salts thereof.

6. The pharmaceutical combination for use according to claim 1, wherein said p38 kinase inhibitor is pamapimod (6-(2,4-Difluorophenoxy)-2-[3-hydroxy-1-(2-hydroxyethyl)-propylamino]-8-methyl-8H-pyrido[2,3-d]pyrimidin-7-one, Formula III) or a pharmaceutically acceptable salt thereof.

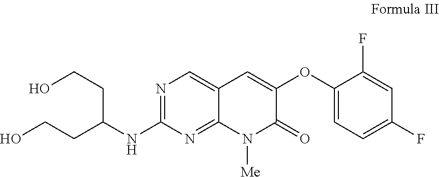 [SMILES: Cn1c(=O)c(Oc2ccc(F)cc2F)cc2cnc(NC(CCO)CCO)nc21]

7. The pharmaceutical combination for use according to any one of claims 1-6, wherein said PPAR agonist is activating PPAR gamma and/or PPAR alpha.

8. The pharmaceutical combination for use according to any one of claims 1-6, wherein said PPAR agonist is activating PPAR gamma.

9. The pharmaceutical combination for use according to any one of claims 1-6, wherein said PPAR agonist is selected from the group consisting of pioglitazone, rosiglitazone troglitazone, fenofibrate, bezafibrate and pharmaceutically acceptable salts thereof.

10. The pharmaceutical combination for use according to any one of claims 1-6, wherein said PPAR agonist is pioglitazone or a pharmaceutically acceptable salt thereof.

11. The pharmaceutical combination for use according to any one of claims 1-10, wherein said viral disease or disorders are caused by viruses affecting the lung.

12. The pharmaceutical combination for use according to any one of claims 1-10, wherein said viral diseases or disorders are caused by a virus selected from the group consisting of SARS-CoV-2 (Coronavirus associated with COVID-19); SARS-CoV(Coronavirus associated with SARS); HCoV (human coronavirus); HA and NA influenza viruses; ADV (adenovirus); HBoV (human bocavirus); HMPV (human metapneumovirus); HPIV (human parainfluenza virus); HRSV (human respiratory syncytial virus); and HRV (human rhinovirus).

13. The pharmaceutical combination for use according to any one of claims 1-10, wherein said viral diseases or disorders are caused by a coronavirus, preferably by SARS-CoV-2.

14. The pharmaceutical combination for use according to any one of claims 1-10, wherein said viral disease or disorder is COVID-19.

15. A kit for use in a method of preventing or treating viral diseases or disorders in a subject, comprising a pharmaceutical combination comprising:
(a) a PPAR agonist;
(b) a p38 kinase inhibitor; and optionally
(c) one or more pharmaceutically acceptable diluents, excipients or carriers; and instructions for using the kit.
